(19) United States
(12) Patent Application Publication
(10) Pub. No.: US 2024/0390246 A1
(43) Pub. Date: Nov. 28, 2024

(54) IMPROVED PERFUME COMPOSITIONS COMPRISING SULFUR-CONTAINING PRO-FRAGRANCE COMPOUNDS

(71) Applicant: Firmenich SA, Satigny (CH)

(72) Inventors: Arnaud STRUILLOU, Satigny (CH); Estelle BOIS, Satigny (CH); Estelle RASSAT, Satigny (CH)

(21) Appl. No.: 18/689,307

(22) PCT Filed: Oct. 20, 2022
(86) PCT No.: PCT/EP22/79226

§ 371 Date: Mar. 5, 2024

(30) Foreign Application Priority Data
Oct. 20, 2021 (EP) 21203749.3

(51) Int. Cl. A61K 8/46; A61K 8/899; A61Q 5/12; A61Q 13/00; A61Q 19/10

(52) U.S. Cl. CPC A61K 8/46; A61K 8/899; A61Q 5/12; A61Q 13/00; A61Q 19/10

(57) ABSTRACT

Described herein is a perfume composition including at least one sulfur-containing pro-fragrance compound and at least one aldehydic perfumery raw material, where the at least one aldehydic perfumery raw material is present in an amount effective to reduce the formation of hydrogen sulfide from the sulfur-containing pro-fragrance. Also described herein are consumer products including the perfume composition as well as the use of at least one aldehydic perfumery raw material for reducing the formation of hydrogen sulfide from a sulfur-containing pro-fragrance.

### TECHNICAL FIELD

The present invention relates to a perfume composition comprising at least one sulfur-containing pro-fragrance compound and at least one aldehydic perfumery raw material, wherein the at least one aldehydic perfumery raw material is present in an amount effective to reduce the formation of hydrogen sulfide from the sulfur-containing pro-fragrance. The present invention further relates to consumer products comprising the perfume composition according to the invention as well as to the use of at least one aldehydic perfumery raw material for reducing the formation of hydrogen sulfide from a sulfur-containing pro-fragrance.

### BACKGROUND OF THE INVENTION

Perfume compositions that comprise sulfur-containing pro-fragrances are widely used in the perfume industry. However, there is risk that at least part of the sulfur-containing pro-fragrances in a perfume composition is oxidized, which leads to the formation of hydrogen sulfide having an unpleasant odor comparable to that of rotten egg.

Several efforts have been made to reduce the formation of hydrogen sulfide in a perfume oil that comprises one or more sulfur-containing pro-fragrances. One approach is to add scavenger molecules to the perfume composition that are capable of trapping the formed hydrogen sulfide, which prevents the unpleasant perception of the hydrogen sulfide. As an example, triethanolamine is frequently used as hydrogen sulfide scavenger molecule that is capable of trapping hydrogen sulfide by forming an odorless salt.

However, when perfume compositions comprising sulfur-containing pro-fragrances are incorporated into consumer products such as home care or personal care products, the above-mentioned concept of hydrogen sulfide scavenging does not work well any more. In particular, in acidic consumer products such as fabric conditioner formulations or shower gels, the trapped hydrogen sulfide can again be released from the scavenger molecule thus leading to the formation of unpleasant rotten egg odor in the final consumer product. Even in some home care or personal care products having a neutral pH (pH value of approximately 7), hydrogen sulfide may be perceivable.

For this reason, preservatives are often added to consumer products such as benzisothiazolin-3-one (BIT), methylchloroisothiazolinone (CIT), or methylisothiazolinone (MIT) to reduce the formation and/or release of hydrogen sulfide in a consumer product. However, such preservatives recently came under regulatory pressure and might need to be removed from such consumer products in the near future.

In view of the above, there is a need to find alternative and/or improved solutions to the above-mentioned problem with regard to the formation of hydrogen sulfide from sulfur-containing pro-fragrance compounds within a perfume composition and within consumer products, respectively.

Within the context of the present invention, it has surprisingly been found that aldehydic perfumery raw materials are capable of reducing the formation of hydrogen sulfide from a sulfur-containing pro-fragrance compound, in particular in perfume compositions. Hence, the present invention is directed to perfume compositions that comprise an amount of aldehydic perfumery raw material(s) that is effective in reducing the formation of hydrogen sulfide from sulfur-containing pro-fragrance compounds.

### DESCRIPTION OF THE FIGURES

FIG. 1. Relative percentage formation of hydrogen sulfide from 3-(dodecylthio)-1-(2,6,6-trimethylcyclohex-3-en-1-yl) butan-1-one (HaloScent® D) in a perfume composition comprising aldehydic perfume raw materials of group A.

FIG. 2. Relative percentage formation of hydrogen sulfide from 3-(dodecylthio)-1-(2,6,6-trimethylcyclohex-3-en-1-yl) butan-1-one (HaloScent® D) in a perfume composition comprising aldehydic perfume raw materials of group B.

FIG. 3. Relative percentage formation of hydrogen sulfide from 3-(dodecylthio)-1-(2,6,6-trimethylcyclohex-3-en-1-yl) butan-1-one (HaloScent® D) in a perfume composition comprising aldehydic perfume raw materials of group C.

### DETAILED DESCRIPTION

The present invention relates to a perfume composition comprising at least one sulfur-containing pro-fragrance compound and at least one aldehydic perfumery raw material, wherein the at least one aldehydic perfumery raw material is present in an amount effective to reduce the formation of hydrogen sulfide from the sulfur-containing pro-fragrance compound, and
wherein the at least one aldehydic perfumery raw material shows a chemical structure selected from the group consisting of R—CH₂—CHO and R′—CH(R′)—CHO;
wherein R represents a hydrogen atom, or a C₁ to C₁₈ hydrocarbon group, optionally comprising one to three oxygen atoms and/or one to two nitrogen atoms and/or one sulfur atom;
wherein R′ groups, independently from each other, represent a C₁ to C₁₈ hydrocarbon group, optionally comprising one to three oxygen atoms and/or one to two nitrogen atoms and/or one sulfur atom; or both R′, when taken together, form a C₅₋₁₆ cycloalkyl, C₅₋₁₆ cycloalkenyl, C₄₋₁₄ heterocycloalkyl or C₄₋₁₄ heterocycloalkenyl group, each optionally substituted with one or more of a C₁₋₁₅ alkyl, C₂₋₁₅ alkenyl, C₁₋₁₅ alkoxy, C₃₋₁₅ cycloalkyl, C₅₋₁₅ cycloalkenyl, C₆₋₁₀ aryl and/or C₆₋₁₀ aryloxy group, each optionally substituted with one or more of a C₁₋₈ alkyl, C₁₋₈ alkoxy, carboxylic acid and/or C₁₋₄ carboxylic ester group, wherein the heteroatom represents one or more of an oxygen atom.

Under a “perfume composition” is to be understood a composition, liquid at about 20° C., that is able to impart a hedonic effect. In other words, a composition to be considered as being a perfume composition must be recognized by a skilled person in the art of perfumery as being able to impart or modify the olfactory perception in a positive or pleasant way, and not just as imparting an odor.

According to the present invention, the perfume composition comprises at least one sulfur-containing pro-fragrance compound.

A pro-perfume or pro-fragrance is a compound that is able to release one, two or three perfume compounds, also termed PRMs (perfumery raw materials), upon external influence in a way that the perfuming effect of the PRMs is prolonged. Thereby, the perfumery raw materials are released from the pro-perfume compound by (chemical) cleavage of the pro-perfume compound. Typically, the pro-perfume itself has a low volatility, and is ideally (almost) odorless. The pro-perfume may be advantageously characterized by a vapor pressure below 0.01 Pa, as obtained by calculation using the software EPIwin v. 3.10 (2000, available at the US Environmental Protection Agency). According to one embodiment, the vapor pressure is below 0.001 Pa. The pro-perfume may also be advantageously characterized by a molecular weight above 270, or above 300, or above 350. The terms “pro-perfume” or “pro-fragrance” have the normal meaning in the art as for example reported in A. Herrmann, Angew. Chem. Int. Ed., 2007, 46, 5836-5863.

The external influence leading to the cleavage of the pro-perfume compound may be light. By “light”, any form of electromagnetic radiation is meant, which is not limited to any particular wavelength. The release of PRMs from such a pro-perfume compound is usually more effective at lower wavelengths (higher energy input).

The cleavage of a certain pro-perfume compound may also be triggered by air/oxygen. Thereby, the PRMs may be released from the pro-perfume compound by oxidation in the presence of air (ambient air) or oxygen.

Moreover, the PRMs may be released from a certain pro-perfume compound by heat. By “heat”, it is meant any energy input that is caused by increased temperature.

Further, the PRMs may be released from a certain pro-perfume compound by moisture. Such a pro-perfume compound may show chemical bonds that are susceptible to water-induced cleavage and may thus be cleaved in the presence of water. In some cases, a certain pH-value may induce and/or support the cleavage.

Further, the PRMs may be released from a certain pro-perfume compound upon exposure to enzymes. Such a pro-perfume compound may show chemical bonds that can efficiently be cleaved in the presence of enzymes.

In some cases, the PRMs may be released from a certain pro-perfume compound not only based on one type of release mechanism but based on two or more of the above-mentioned types simultaneously, such as for example release by air/oxygen and moisture.

Since, according to the invention, the perfume composition comprises at least one sulfur-containing pro-fragrance compound, said sulfur-containing pro-fragrance compound is at risk of forming unpleasant hydrogen sulfide upon oxidation of said compound.

In a particular embodiment, the perfume composition comprises one, two, three, four, or more sulfur-containing pro-fragrance compounds. Preferably, the perfume composition comprises one or two sulfur-containing pro-fragrance compounds. More preferably, the perfume composition comprises one sulfur-containing pro-fragrance compound.

In a particular embodiment, the sulfur-containing pro-fragrance compound is of formula

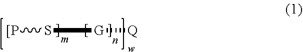

wherein:
a) w represents an integer from 1 to 10000;
b) n represents 1 or 0;
c) m represents an integer from 1 to 6;
d) P represents a hydrogen atom or a radical susceptible of generating an odoriferous α,β-unsaturated ketone, aldehyde or carboxylic ester and is represented by the formula

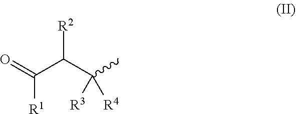

in which the wavy line indicates the location of the bond between said P and S; R¹ represents a hydrogen atom, a C₁ to C₆ alkoxyl radical or a C₁ to C₁₅ linear, cyclic or branched alkyl, alkenyl or alkadienyl radical, optionally substituted by one to four C₁ to C₄ alkyl groups; and 20
R², R³ and R⁴ represent independently of each other a hydrogen atom, an aromatic ring, or a C₁ to C₁₅ linear, cyclic or branched alkyl, alkenyl or alkadienyl radical, possibly substituted by C₁ to C₄ alkyl groups; or two, or three, of the groups R¹ to R⁴ are bonded together to form a saturated or unsaturated ring having 5 to 20 carbon atoms and including the carbon atom to which said R¹, R², R³ or R⁴ groups are bonded, this ring being possibly substituted by C₁ to C₈ linear, branched or cyclic alkyl or alkenyl groups; and with the proviso that at least one of the P groups is of the formula (II) as defined hereinabove;
e) G represents a multivalent radical (with a m+1 valence) derived from cyclic, linear, alicyclic or branched alkyl, cyclic, linear, alicyclic or branched alkenyl, phenyl, alkylphenyl or alkenylphenyl hydrocarbon radical having from 1 to 22 carbon atoms, said hydrocarbon radical being possibly substituted and containing from 1 to 10 functional groups selected from the group consisting of halogens, alcohols, ethers, esters, ketones, aldehydes, carboxylic acids, thiols, thioethers, amines, quaternary amines and amides; and
f) Q represents a hydrogen atom (in which case w=1 and n=1), or represents a polymer or co-polymer selected from the group consisting of poly(alkylimine)s, peptides (e.g. lysine) or polysaccharides selected from the group consisting of cellulose, cyclodextrins and starches, or cationic quaternised silicon polymers, or still a polymer or random co-polymer derived from monomeric units selected from the group consisting of the formulae A-1), A-2), A-3), B-1), B-2), C-1), C-2), and C-3):

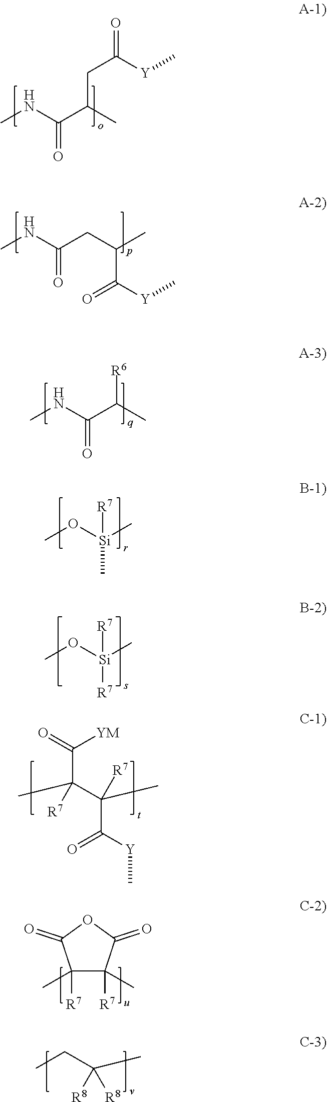

wherein the hatched lines indicate the location of the bond between said monomeric unit and G;
Y represents an oxygen or sulfur atom or a NR⁷ group;
o, p, q, r, s, t, u and v all represent independent of each other fractions between 0 and 1, with o+p+q=1, r+s=1 and t+u+v=1 and with the proviso that either o or p, as well as r and t are not equal to 0;
R⁶ represents a hydrogen atom or a side chain from a natural or unnatural amino acid, such as glycine, alanine, phenylalanine, arginine, histidine, lysine, aspartic acid, glutamic acid, cysteine, methionine, glutamine, asparagine, threonine, serine, leucine, isoleucine, valine, tyrosine or tryptophan;
R⁷ represents, simultaneously or independently, a hydrogen atom or a C₁-C₁₆ hydrocarbon group;
R⁸ represents, simultaneously or independently of each other
   a hydrogen or halide atom;
   a C₁-C₆ hydrocarbon group optionally comprising from 1 to 4 heteroatoms selected from the group consisting of oxygen and sulfur atoms;
   a carboxylic group of formula COOR*, wherein R* represents a hydrogen atom, a C₁-C₆₀ alkyl or alkenyl group optionally comprising from 1 to 30 oxygen atoms;
   a OR⁷ group or a COR⁷ group; or
   a pyrrolidone unit, connected by the nitrogen atom; and
M represents a hydrogen atom, an alkali or earth alkali metal ion.

As “odoriferous α,β-unsaturated ketone, aldehyde or carboxylic ester”, the expression used in the definition of P, it is understood an α,β-unsaturated ketone, aldehyde or carboxylic ester, which is recognized by a skilled person as being used in perfumery as perfuming ingredient. In general, said odoriferous α,β-unsaturated ketone, aldehyde or carboxylic ester is a compound having from 8 to 20 carbon atoms, or even more preferably between 10 and 15 carbon atoms.

Similarly, it is not possible to provide an exhaustive list of the currently known odoriferous compounds, which can be used in the synthesis of compounds of formula (I) defined hereinabove and subsequently be released. However, the following can be named as preferred examples: alpha-damascone, beta-damascone, gamma-damascone, delta-damascone, alpha-ionone, beta-ionone, gamma-ionone, delta-ionone, beta-damascenone, 2-methyl-1-(2,6,6-trimethylcyclohex-3-en-1-yl) but-2-en-1-one, 1-[6-ethyl-2,6-dimethyl-3-cyclohexen-1-yl]-2-buten-1-one, 3-methyl-5-propyl-2-cyclohexen-1-one, 2-methyl-5-(1-propen-2-yl)-2-cyclohexen-1-one, 2,5-dimethyl-5-phenyl-1-hexen-3-one, 1-(5,5-dimethyl-1-cyclohexen-1-yl)-4-penten-1-one, 3,7-dimethylocta-2,6-dienal, 8-methyl-alpha-ionone or 10-methyl-alpha-ionone, 2-octenal, 1-(2,2,3,6-tetramethylcyclohexyl) but-2-en-1-one, 4-(2,2,3,6-tetramethylcyclohexyl) but-3-en-2-one, 2-cyclopentadecen-1-one, 4,4a-dimethyl-6-(1-propen-2-yl)-4,4a,5,6,7,8-hexahydro-2 (3H)-naphthalenone, cinnamic aldehyde, 2,6,6-trimethylspiro[bicyclo[3.1.1]heptane-3,1′-cyclohexan]-2′-en-4′-one, ethyl 2,4-deca-dienoate, ethyl 2-octenoate, methyl 2-nonenoate, ethyl 2,4-undecadienoate, 4-methylpent-3-en-2-one, oct-2-en-4-one, and methyl 5,9-dimethyl-2,4,8-decatrienoate.

In a particular embodiment, P represents a radical selected from the group consisting of formulae (P-1) to (P-14), in the form of any one of its isomers:

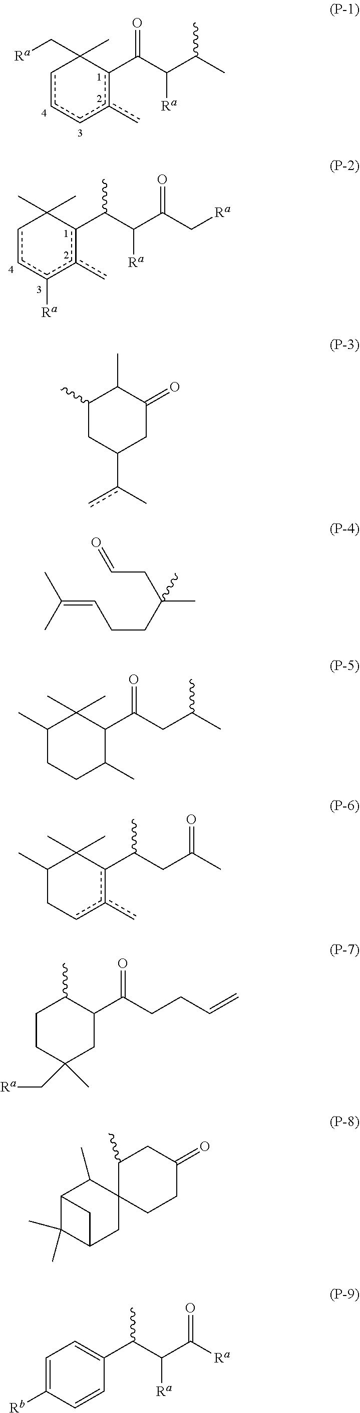

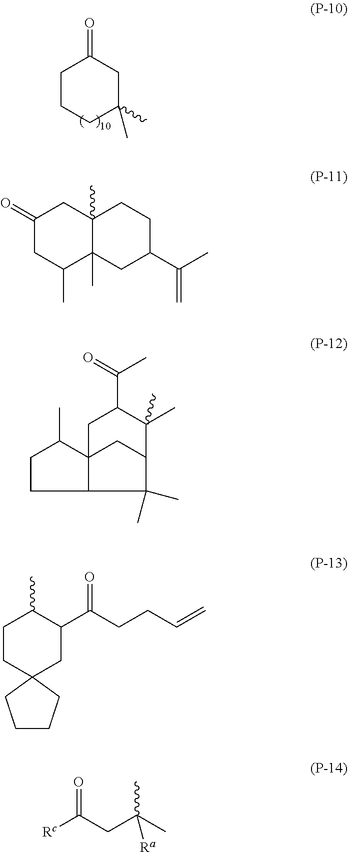

in which formulae the wavy lines have the meaning indicated above and the dotted lines represent a single or double bond, Ra being a hydrogen atom or a methyl group and Rb representing a hydrogen atom, a hydroxyl or methoxy group or a C₁-C₄ linear or branched alkyl group and R^{c} representing a hydrogen atom or a C₁-C₄ linear or branched alkyl group.

In a particular embodiment, P represents a radical selected from the group consisting of formulae

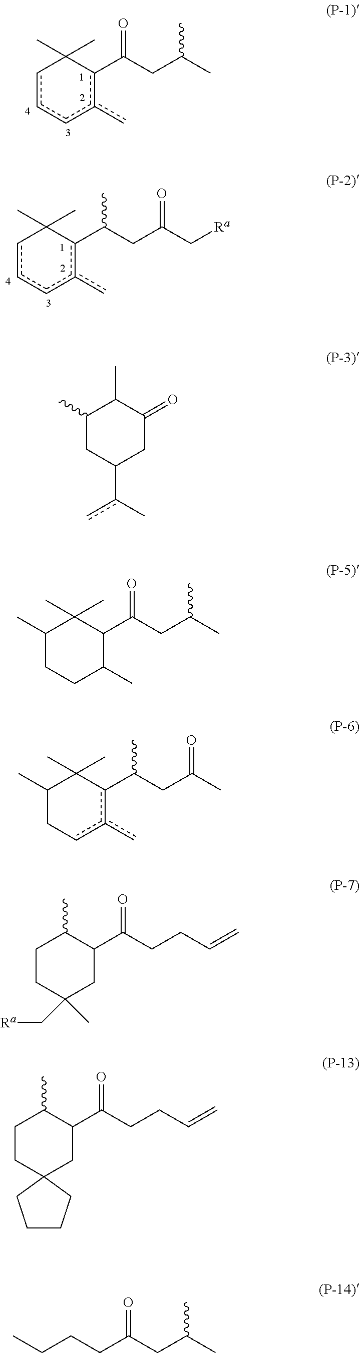

wherein the wavy lines have the meaning indicated above and the dotted lines represent a single or double bond, and Ra being a hydrogen atom or a methyl group.

In a particular embodiment, P represents a radical selected from the group consisting of formulae (P-1), (P-2), (P-1)′, (P-2)′, (P-3), (P-7), (P-13), (P-14) or (P-14)′ as defined above. Preferably, P represents a radical selected from the group consisting of formulae (P-1), (P-1)′, (P-2), (P-2)′, (P-3) or (P-14)′ as defined above.

In a particular embodiment, G may represent a divalent cyclic, linear or branched alkyl, alkenyl, alkandienyl or alkylbenzene hydrocarbon radical having from 1 to 22 carbon atoms, said hydrocarbon radical being possibly substituted and containing from 1 to 10 functional groups selected from the group consisting of ethers, esters, ketones, aldehydes, carboxylic acids, thiols, thioethers, amines, quaternary amines and amides.

In a particular embodiment, G represents a divalent linear or branched alkyl hydrocarbon radical having from 1 to 22 carbon atoms, said hydrocarbon radical being possibly substituted and containing from 1 to 5 functional groups selected from the group consisting of ethers, esters, ketones, aldehydes, carboxylic acids, thiols, thioethers, amines, quaternary amines and amides.

In a particular embodiment, G represents a divalent linear or branched alkyl hydrocarbon radical having from 2 to 15 carbon atoms, said hydrocarbon radical being possibly substituted and containing from 1 to 2 functional groups selected from the group consisting of ethers and esters.

In a particular embodiment, G represents a divalent linear alkyl hydrocarbon radical having from 3 to 15 carbon atoms, said hydrocarbon radical being possibly substituted and containing one ester functional group.

In a particular embodiment, G represents a divalent linear alkyl hydrocarbon radical having from 3 to 14 carbon atoms.

In a particular embodiment, Q represents a hydrogen atom or a co-polymer comprising at least one repeating unit of formula B-1 as defined above.

In a particular embodiment, Q represents a hydrogen atom or a co-polymer comprising at least one repeating unit of formula B-1 and at least one repeating unit of formula B-2.

In a particular embodiment, R⁷ represents, simultaneously or independently, a hydrogen atom or a C₁₋₃ alkyl group. Preferably, R⁷ represents, simultaneously or independently, a hydrogen atom or a methyl or an ethyl group. More preferably, R⁷ represents, simultaneously or independently, a hydrogen atom or a methyl group.

In a particular embodiment, the pro-perfume compound is defined by formula (I) as mentioned above, wherein
w=1; n=1; m=1;
P represents a radical susceptible of generating an odoriferous α,β-unsaturated ketone, aldehyde and is represented by the formula

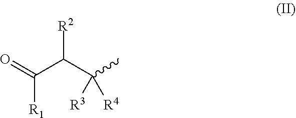

wherein R², R³ and R⁴ represent independently of each other a hydrogen atom, a C₆ to C₁₀ aromatic ring, or a C₁ to C₁₅ linear, cyclic or branched alkyl, alkenyl or alkadienyl radical, possibly substituted by C₁ to C₄ alkyl groups; or two, or three, of the groups R¹ to R⁴ are bonded together to form a saturated or unsaturated ring having 5 to 20 carbon atoms and including the carbon atom to which said R¹, R², R³ or R⁴ groups are bonded, this ring being possibly substituted by C₁ to C₈ linear, branched or cyclic alkyl or alkenyl groups;
G represents a divalent radical derived from cyclic, linear or branched alkyl, alkenyl, phenyl, alkylphenyl or alkenylphenyl hydrocarbon radical having from 2 to 8 carbon atoms optionally comprising 1 or 2 oxygen, sulfur and/or nitrogen atoms
Q represents a polymer or random co-polymer derived from formula B-1), wherein R⁷ represents a C₁-C₁₆ hydrocarbon group.

In a particular embodiment, the sulfur-containing pro-perfume compound is a linear polysiloxane co-polymer comprising at least one repeating unit of formula

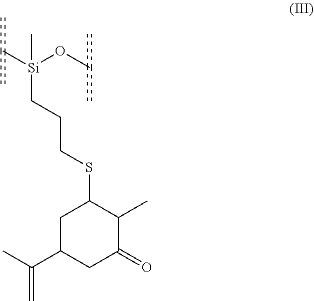

wherein the double hatched lines indicate the bonding to another repeating unit.

The pro-perfume of formula (III) releases 2-methyl-5-(prop-1-en-2-yl)cyclohex-2-en-1-one as fragrance compound, which is also known as carvone. Carvone exists in the form of two enantiomers, namely (R)-(−)-2-methyl-5-(1-propen-2-yl)-2-cyclohexen-1-one (I-carvone or carvone laevo) and(S)-(+)-2-methyl-5-(1-propen-2-yl)-2-cyclohexen-1-one (d-carvone or carvone dextro). The two enantiomers have been reported to have slightly different mint odor tonalities. Nevertheless, both enantiomers are expected to have a similar effect in view of the preparation of the co-polymer and the release efficiency.

In a particular embodiment, the sulfur-containing pro-perfume compound is of formula (IV)

P—S-G-Q (IV)

wherein
P has the same meaning as defined above:
G represents a divalent radical derived from a linear or branched alkyl or alkenyl radical having from 2 to 15 carbon atoms, possibly substituted with one or more groups selected from the group consisting of —OR⁹, —(NR⁹)₂, —COOR⁹ and R⁹ groups, in which R⁹ represents a hydrogen atom or a C₁ to C₆ alkyl or alkenyl group; and
Q represents a hydrogen atom.

In a particular embodiment, the pro-perfume compound of formula (I) is a compound of formulae a) to d)

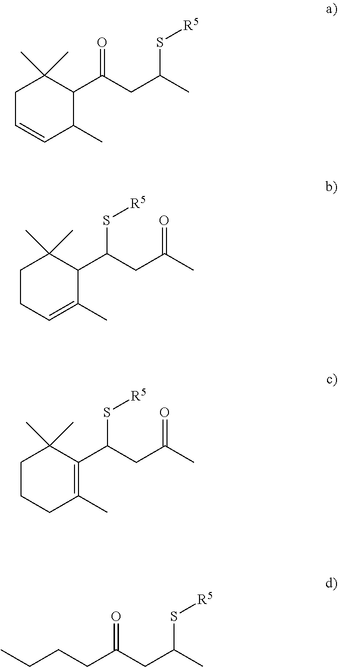

or any combination thereof;

wherein R⁵ represents a C₁-C₂₀ alkyl or alkenyl group, preferably a C₆-C₁₅ alkyl or alkenyl group, more preferably a C₁₂ alkyl group.

In a particular embodiment, the pro-fragrance compound of formula (I) may be selected from the group consisting of methyl or ethyl 2-(4-oxo-4-(2,6,6-trimethylcyclohex-3-en-1-yl) butan-2-ylamino)-3-(4-oxo-4-(2,6,6-trimethylcyclohex-3-en-1-yl) butan-2-ylthio) propanate, methyl or ethyl 2-(4-oxo-4-(2,6,6-trimethylcyclohex-2-en-1-yl) butan-2-ylamino)-3-(4-oxo-4-(2,6,6-trimethylcyclohex-2-en-1-yl) butan-2-ylthio) propanate, methyl or ethyl 2-(2-oxo-4-(2,6,6-trimethylcyclohex-1-en-1-yl) butan-4-ylamino)-3-(2-oxo-4-(2,6,6-trimethylcyclohex-1-en-1-yl) butan-4-ylthio) propanate, methyl or ethyl 2-(2-oxo-4-(2,6,6-trimethylcyclohex-2-en-1-yl) butan-4-ylamino)-3-(2-oxo-4-(2,6,6-trimethylcyclohex-2-en-1-yl) butan-4-ylthio) propanate, 3-(dodecylthio)-1-(6-ethyl-2,6-dimethylcyclohex-3-en-1-yl) butan-1-one, 3-(dodecylthio)-1-(2,6,6-trimethylcyclohex-3-en-1-yl)-1-butanone, 3-(dodecylthio)-1-(2,6,6-trimethylcyclohex-2-en-1-yl)-1-butanone, 4-(dodecylthio)-4-(2,6,6-trimethylcyclohex-2-en-1-yl)-2-butanone, 4-(dodecylthio)-4-(2,6,6-trimethylcyclohex-1-en-1-yl)-2-butanone, 2-dodecylsulfanyl-5-methyl-heptan-4-one, 2-cyclohexyl-1-dodecylsulfanyl-hept-6-en-3-one, 3-(dodecylthio)-5-isopropenyl-2-methylcyclohexanone, 2-(dodecylthio)-4-octanone, 4-(dodecylthio)-4-methylpentan-2-one, methyl or ethyl N,S-bis(4-oxo-4-(2,6,6-trimethylcyclohex-3-en-1-yl) butan-2-yl)-L-cysteinate, methyl or ethyl S-(4-oxo-4-(2,6,6-trimethylcyclohex-3-en-1-yl) butan-2-yl)-L-cysteinateand any mixtures thereof.

Preferably, the pro-fragrance compound of formula (I) is selected from the group consisting of 3-(dodecylthio)-1-(2,6,6-trimethylcyclohex-3-en-1-yl) butan-1-one (HaloScent® D), 3-(dodecylthio)-1-(2,6,6-trimethylcyclohex-2-en-1-yl) butan-1-one, 4-(dodecylthio)-4-(2,6,6-trimethylcyclohex-2-en-1-yl) butan-2-one (HaloScent® I) and 4-(dodecylthio)-4-(2,6,6-trimethylcyclohex-1-en-1-yl) butan-2-one (HaloScent® I), 2-(dodecylthio)-4-octanone, 2-(dodecylsulfonyl) octan-4-one, 4-(dodecylthio)-4-methylpentan-2-one, methyl or ethyl N,S-bis(4-oxo-4-(2,6,6-trimethylcyclohex-3-en-1-yl) butan-2-yl)-L-cysteinate, methyl or ethyl S-(4-oxo-4-(2,6,6-trimethylcyclohex-3-en-1-yl) butan-2-yl)-L-cysteinate and any mixtures thereof. Preferably, the profragrance compound of formula (I) may be 3-(dodecylthio)-1-(2,6,6-trimethylcyclohex-3-en-1-yl) butan-1-one (HaloScent® D), 4-(dodecylthio)-4-(2,6,6-trimethylcyclohex-2-en-1-yl) butan-2-one (HaloScent® I), 4-(dodecylthio)-4-(2,6,6-trimethylcyclohex-1-en-1-yl) butan-2-one (HaloScent® I), or a mixture thereof. Preferably, the pro-fragrance compound of formula (I) is a mixture of 3-(dodecylthio)-1-(2,6,6-trimethylcyclohex-3-en-1-yl) butan-1-one (HaloScent® D), 4-(dodecylthio)-4-(2,6,6-trimethylcyclohex-1-en-1-yl) butan-2-one (HaloScent® I) and 4-(dodecylthio)-4-(2,6,6-trimethylcyclohex-2-en-1-yl) butan-2-one (HaloScent® I).

In another preferred embodiment, the sulfur-containing pro-fragrance compound of formula (I) is 3-(dodecylthio)-1-(2,6,6-trimethylcyclohex-3-en-1-yl) butan-1-one (HaloScent® D).

In another preferred embodiment, the sulfur-containing pro-fragrance compound of formula (I) is a mixture of 4-(dodecylthio)-4-(2,6,6-trimethylcyclohex-1-en-1-yl) butan-2-one (HaloScent® I) and 4-(dodecylthio)-4-(2,6,6-trimethylcyclohex-2-en-1-yl) butan-2-one (HaloScent® I).

In a particular embodiment, the perfume composition comprises the at least one sulfur-containing pro-fragrance compound in an amount of at least 0.05 wt. %, preferably at least 0.1 wt. %, more preferably at least 1 wt. % and even more preferably at least 2 wt. %, based on the total weight of the perfume composition.

In another particular embodiment, the perfume composition comprises the at least one sulfur-containing pro-fragrance compound in an amount of at least 5 wt. %, preferably at least 10 wt. %, more preferably at least 15 wt. %.

In a particular embodiment, the perfume composition comprises the at least one sulfur-containing pro-fragrance compound in an amount from 0.05 to 50 wt. %, preferably in an amount from 0.1 to 30 wt. %, more preferably in an amount from 1 to 25 wt. %, more preferably in an amount from 2 to 25 wt. %, even more preferably in an amount from 2 to 15 wt. %, based on the total weight of the perfume composition.

According to the invention, the perfume composition comprises at least one aldehydic perfumery raw material, wherein the at least one aldehydic perfumery raw material shows a chemical structure selected from the group consisting of R—CH₂—CHO and R′—CH(R′)—CHO.

It is understood that by “hydrocarbon group” it is meant that said group consists of hydrogen and carbon atoms and can be in the form of an aliphatic hydrocarbon, i.e. linear or branched saturated hydrocarbon (e.g. alkyl group), a linear or branched unsaturated hydrocarbon (e.g. alkenyl or alkynyl group), a saturated cyclic hydrocarbon (e.g. cycloalkyl) or an unsaturated cyclic hydrocarbon (e.g. cycloalkenyl or cycloalkynyl), or can be in the form of an aromatic hydrocarbon, i.e. aryl group, or can also be in the form of a mixture of said type of groups, e.g. a specific group may comprise a linear alkyl, a branched alkenyl (e.g. having one or more carbon-carbon double bonds), a (poly)cycloalkyl and an aryl moiety, unless a specific limitation to only one type is mentioned. Similarly, in all the embodiments of the invention, when a group is mentioned as being in the form of more than one type of topology (e.g. linear, cyclic or branched) and/or being saturated or unsaturated (e.g. alkyl, aromatic or alkenyl), it is also meant a group which may comprise moieties having any one of said topologies or being saturated or unsaturated, as explained above. Similarly, in all the embodiments of the invention, when a group is mentioned as being in the form of one type of saturation or unsaturation, (e.g. alkyl), it is meant that said group can be in any type of topology (e.g. linear, cyclic or branched) or having several moieties with various topologies.

The term “optionally” is understood that a certain group to be optionally substituted or optionally comprising can or cannot be substituted with a certain functional group or can or cannot comprise a certain atom. The term “one or more” is understood as being substituted with 1 to 7, preferably 1 to 5 and more preferably 1 to 3 of a certain functional group.

The terms “alkyl” and “alkenyl” are understood as comprising branched and linear alkyl and alkenyl groups. The terms “alkenyl”, “cycloalkenyl” and “heterocycloalkenyl” is understood as comprising 1, 2 or 3 olefinic double bonds, preferably 1 or 2 olefinic double bonds. The terms “cycloalkyl”, “cycloalkenyl”, “heterocycloalkyl”, “heterocycloalkenyl” and “heterocyclic” are understood as comprising a monocyclic or fused, spiro and/or bridged bicyclic or tricyclic cycloalkyl, cycloalkenyl, heterocycloalkyl, heterocycloalkenyl and heterocyclic groups, preferably monocyclic cycloalkyl, cycloalkenyl, heterocycloalkyl and heterocycloalkenyl groups.

The term “aryl” is understood as comprising any group comprising at least one aromatic group such as phenyl, indenyl, indanyl, benzodioxolyl, dihydrobenzodioxinyl, tetrahydronaphthalenyl or naphthalenyl group.

A “perfume compound” or “perfumery raw material” is a compound, which is used as an active ingredient in perfume preparations or compositions in order to impart a hedonic effect; i.e. it is used for the primary purpose of conferring or modulating a pleasant odor. In other words, a compound to be considered as being a perfume compound must be recognized by a skilled person in the art of perfumery as being able to impart or modify the odor of a composition in a positive or pleasant way, and not just as having an odor.

According to the invention, the aldehydic perfumery raw material is not an aldehydic pro-fragrance compound.

In a particular embodiment, the perfume composition comprises one, two, three, four, or more of the aldehydic perfumery raw materials. In an embodiment, the perfume composition comprises one aldehydic perfumery raw material. In another embodiment, the perfume composition comprises two aldehydic perfumery raw materials. In another embodiment, the perfume composition comprises three aldehydic perfumery raw materials. In another embodiment, the perfume composition comprises four aldehydic perfumery raw materials.

In another embodiment, the perfume composition comprises five aldehydic perfumery raw materials. In another embodiment, the perfume composition comprises more than five aldehydic perfumery raw materials.

According to the invention, R may be a hydrogen atom, or a C₁ to C₁₆ hydrocarbon group, optionally comprising one to three oxygen atoms and/or one to two nitrogen atoms and/or one sulfur atom. Preferably, R may be a hydrogen atom, or a C₁ to C₁₄ hydrocarbon group, optionally comprising one to three oxygen atoms and/or one to two nitrogen atoms and/or one sulfur atom. Preferably, R may be a hydrogen atom, or a C₁ to C₁₂ hydrocarbon group, optionally comprising one to three oxygen atoms and/or one to two nitrogen atoms and/or one sulfur atom. Preferably, R may be a hydrogen atom, or a C₁ to C₁₀ hydrocarbon group, optionally comprising one to three oxygen atoms and/or one to two nitrogen atoms and/or one sulfur atom. Preferbaly, R may be a C₁ to C₁₀ alkyl or a C₂ to C₁₀ alkenyl group, Preferbaly, R may be a C₃ to C₁₀ alkyl or a C₃ to C₁₀ alkenyl group. Preferbaly, R may be a C₅ to C₁₀ alkyl or a C₃ to C₁₀ alkenyl group. Even more preferably, R may be a C₆ to C₁₀ alkyl or a C₆ to C₁₀ alkenyl group.

According to the invention, the first R′ group may be a C₁ to C₁₆ hydrocarbon group, optionally comprising one to three oxygen atoms and/or one to two nitrogen atoms and/or one sulfur atom. Preferably, the first R′ group may be a C₁ to C₁₄ hydrocarbon group, optionally comprising one to three oxygen atoms and/or one to two nitrogen atoms and/or one sulfur atom. Preferably, the first R′ group may be a C₁ to C₁₂ hydrocarbon group, optionally comprising one to three oxygen atoms and/or one to two nitrogen atoms and/or one sulfur atom. Preferably, the first R′ group may be a C₁ to C₁₀ hydrocarbon group, optionally comprising one to three oxygen atoms and/or one to two nitrogen atoms and/or one sulfur atom. Preferbaly, the first R′ group may be a C₁ to C₁₀ alkyl or a C₂ to C₁₀ alkenyl group, Preferbaly, the first R′ group may be a C₁ to C₈ alkyl or a C₂ to C₈ alkenyl group. Preferbaly, the first R′ group may be a C₁ to C₆ alkyl or a C₂ to C₆ alkenyl group. Preferably, the first R′ group may be a C₁ to C₄ alkyl or a C₂ to C₄ alkenyl group. Preferably, the first R′ group may be a C₁ to C₃ alkyl or a C₂ to C₃ alkenyl group. Preferably, the first R′ group may be a C₁ to C₂ alkyl or a C₂ alkenyl group. Preferably, the first R′ group may be a methyl group.

According to the invention, the second R′ group may be a C₁ to C₁₆ hydrocarbon group, optionally comprising one to three oxygen atoms and/or one to two nitrogen atoms and/or one sulfur atom. Preferably, the second R′ group may be a C₁ to C₁₄ hydrocarbon group, optionally comprising one to three oxygen atoms and/or one to two nitrogen atoms and/or one sulfur atom. Preferably, the second R′ group may be a C₁ to C₁₂ hydrocarbon group, optionally comprising one to three oxygen atoms and/or one to two nitrogen atoms and/or one sulfur atom. Preferably, the second R′ group may be a C₁ to C₁₀ hydrocarbon group, optionally comprising one to three oxygen atoms and/or one to two nitrogen atoms and/or one sulfur atom. Preferbaly, the second R′ group may be a C₁ to C₁₀ alkyl or a C₂ to C₁₀ alkenyl group, Preferbaly, the second R′ group may be a C₃ to C₁₀ alkyl or a C₃ to C₁₀ alkenyl group. Preferbaly, the second R′ group may be a C₅ to C₁₀ alkyl or a C₃ to C₁₀ alkenyl group. Even more preferably, the second R′ group may be a C₆ to C₁₀ alkyl or a Ce to C₁₀ alkenyl group.

According to the invention, both R′, when taken together, may form a C₅₋₁₄ cycloalkyl, C₅₋₁₄ cycloalkenyl, C₄₋₁₂ heterocycloalkyl or C₄₋₁₂ heterocycloalkenyl group, each optionally substituted with one or more of a C₁₋₁₀ alkyl, C₂₋₁₀ alkenyl, C₁₋₁₀ alkoxy, C₃₋₁₀ cycloalkyl, C₅-cycloalkenyl, C₆₋₈ aryl and/or C₆₋₈ aryloxy group, each optionally substituted with one or more of a C₁₋₆ alkyl, C₁₆ alkoxy, carboxylic acid and/or C₁₋₃ carboxylic ester group, wherein the heteroatom represents one or more of an oxygen atom. Preferably, both R′, when taken together, may form a C₅₋₁₂ cycloalkyl, C₅₋₁₂ cycloalkenyl, C₄₋₁₀ heterocycloalkyl or C₄₋₁₀ heterocycloalkenyl group, each optionally substituted with one or more of a C₁₋₈ alkyl, C₂₋₈ alkenyl, C₁₋₈ alkoxy, C₃₋₈ cycloalkyl, C₅₋₈ cycloalkenyl, C₆ aryl and/or C₆ aryloxy group, each optionally substituted with one or more of a C₁₋₄ alkyl, C₁₋₄ alkoxy, carboxylic acid and/or C₁₋₂ carboxylic ester group, wherein the heteroatom represents one or more of an oxygen atom. Preferably, both R′, when taken together, may form a C₅₋₁₀ cycloalkyl, C₅₋₁₀ cycloalkenyl, C₄₋₈ heterocycloalkyl or C₄₋₈ heterocycloalkenyl group, each optionally substituted with one or more of a C₁₆ alkyl, C₂₋₆ alkenyl, C₁₆ alkoxy, C₃₋₆ cycloalkyl, C₅₋₇ cycloalkenyl, C₆ aryl and/or C₆ aryloxy group, each optionally substituted with one or more of a C₁₄ alkyl, C₁₋₄ alkoxy, carboxylic acid and/or C₁₋₂ carboxylic ester group, wherein the heteroatom represents one or more of an oxygen atom. Preferably, both R′, when taken together, may form a C₅₋₁₀ cycloalkyl, C₅₋₁₀ cycloalkenyl, C₄₋₈ heterocycloalkyl or C₄₋₈ heterocycloalkenyl group, each optionally substituted with one or more of a C₁₋₆ alkyl, C₂₋₆ alkenyl, C₁₆ alkoxy, C₃₋₆ cycloalkyl, C₅₋₇ cycloalkenyl, C₆ aryl and/or C₆ aryloxy group, each optionally substituted with one or more of a C₁₋₃ alkyl, C₁₋₃ alkoxy, carboxylic acid and/or C₁₋₂ carboxylic ester group, wherein the heteroatom represents one or more of an oxygen atom. Preferably, both R′, when taken together, may form a C₅₋₁₀ cycloalkyl, C₅₋₁₀ cycloalkenyl, C₄₋₈ heterocycloalkyl or C₄₋₈ heterocycloalkenyl group, each optionally substituted with one or more of a C₁₄ alkyl, C₂₋₄ alkenyl, C₁₄ alkoxy, C₅₋₆ cycloalkyl, C₅₋₆ cycloalkenyl, C₆ aryl and/or C₆ aryloxy group, each optionally substituted with one or more of a C₁₄ alkyl, C₁₋₄ alkoxy, carboxylic acid and/or C₁₋₂ carboxylic ester group, wherein the heteroatom represents one or more of an oxygen atom. Preferably, both R′, when taken together, may form a C₅₋₈ cycloalkyl, C₅₋₈ cycloalkenyl, C₄₋₆ heterocycloalkyl or C₄₋₆ heterocycloalkenyl group, each optionally substituted with one or more of a C₁₄ alkyl, C₂₋₄ alkenyl, C₁₄ alkoxy, C₅₋₆ cycloalkyl, C₅₋₆ cycloalkenyl, C₆ aryl and/or C₆ aryloxy group, each optionally substituted with one or more of a C₁₋₄ alkyl, C₁₋₄ alkoxy, carboxylic acid and/or C₁₋₂ carboxylic ester group, wherein the heteroatom represents one or more of an oxygen atom.

In a preferred embodiment, the at least one aldehydic perfumery raw material shows a chemical structure of R—CH₂—CHO or R′—CH (Me) CHO, preferably, R—CH₂—CHO.

In a particular embodiment, the at least one aldehydic perfumery raw material is selected from the group consisting of (+/−)-2-methyl-3-[4-(2-methyl-2-propanyl)phenyl]propanal, (+/−)-2-methylundecanal, 2,4-dimethyl-3-cyclohexene-1-carbaldehyde, (+/−)-3-(4-isopropylphenyl)-2-methylpropanal, (+/−)-3-(1,3-benzodioxol-5-yl)-2-methylpropanal, (+/−)-3/4-(4-methyl-3-penten-1-yl)-3-cyclohexene-1-carbaldehyde, (E)-2-dodecenal, (Z)-3-dodecenal, dodecanal, (+/−)-2,6-dimethyl-5-heptenal, (+/−)-3/4-(4-hydroxy-4-methylpentyl)-3-cyclohexene-1-carbaldehyde, (+/−)-3-(4-methoxyphenyl)-2-methylpropanal, (+/−)-8alpha/9alpha-methoxy-1alpha,2beta, 6beta-tricyclo[5.2.1.02.6]decane-3beta-carbaldehyde, 3,5,6-trimethyl-3-cyclohexene-1-carbaldehyde, 2,4,6-trimethyl-3-cyclohexene-1-carbaldehyde, (+/−)-2-methyldecanal, (+/−)-2,6,10-trimethyl-9-undecenal, (1RS,6RS)-3,6-dimethyl-3-cyclohexene-1-carbaldehyde, (1RS,6RS)-4,6-dimethyl-3-cyclohexene-1-carbaldehyde, (1RS,6SR)-4,6-dimethyl-3-cyclohexene-1-carbaldehyde, (+/−)-8,8-dimethyl-1,2,3,4,5,6,7,8-octahydro-2-naphthalenecarbaldehyde, (+/−)-5,5-dimethyl-1,2,3,4,5,6,7,8-octahydro-2-naphthalenecarbaldehyde, (+/−)-2,2-dimethyltricyclo[6.2.1.0^{1,6}]undecan-7-one, 3-(1, 1/3,3-dimethyl-2,3-dihydro-1H-inden-5-yl) propanal, decenal, dodecanal, undecanal, octanal, (+/−)-7-hydroxy-3,7-dimethyloctanal, 10-undecenal, nonanal, 10-undecenal, (9E)-9-undecenal, (9Z)-9-undecenal, 3-(1,1/3,3-dimethyl-2,3-dihydro-1H-inden-5-yl) propanal, (+/−)-3-(3-isopropyl-1-phenyl) butanal, 3-(4-tert-butylphenyl) propanal, (+)—(R)-3,7-dimethyl-6-octenal, (+/−)-3,7-dimethyl-6-octenal, (+/−)-3-phenylbutanal, 3-[(1R)-4-methyl-3-cyclohexen-1-yl]butanal, (4E)-4-methyl-5-(4-methylphenyl)-4-pentenal, 3-(4,4-dimethyl-1-cyclohexen-1-yl) propanal, phenylacetaldehyde, hexanal, (E)-4-decenal, 7-hydroxy-3,7-dimethyloctanal, (4Z)-4-dodecenal, tridecanal, heptanal, 3-phenylpropanal, 3-methylbutanal, (4-methylphenyl) acetaldehyde, 6-methoxy-2,6-dimethyloctanal, 7-isopropyl-5-methylbicyclo[2.2.2]oct-5-ene-2-carbaldehyde, 8-isopropyl-6-methylbicyclo[2.2.2]oct-5-ene-2-carbaldehyde, 3-(4-isobutylphenyl)-2-methylpropanal, octahydro-1H-4,7-methanoindene-2-carbaldehyde, (E)-2,6,10-trimethylundeca-5,9-dienal, (E)-3,7,11-trimethyldodeca-6,10-dienal, 4-(octahydro-5H-4,7-methanoinden-5-ylidene) butanal, 3-(4-isopropylphenyl) propanal, 4,8-dimethyldeca-4,9-dienal, (E)-5,9-dimethyldeca-4,8-dienal, 2-((3,7-dimethylocta-2,6-dien-1-yl)oxy) acetaldehyde, (4Z,7Z)-deca-4,7-dienal, (S,Z)-6-(2,2,3-trimethylcyclopent-3-en-1-yl) hex-4-enal, 6,8-dimethylnon-7-enal and any mixture thereof.

In a particular embodiment, the at least one aldehydic perfumery raw material is selected from the group consisting of (+/−)-2-methyl-3-[4-(2-methyl-2-propanyl)phenyl]propanal, (+/−)-2-methylundecanal, 2,4-dimethyl-3-cyclohexene-1-carbaldehyde, (+/−)-3-(4-isopropylphenyl)-2-methylpropanal, (+/−)-3-(1,3-benzodioxol-5-yl)-2-methylpropanal, (+/−)-3/4-(4-methyl-3-penten-1-yl)-3-cyclohexene-1-carbaldehyde, (E)-2-dodecenal, (Z)-3-dodecenal, dodecanal, (+/−)-2,6-dimethyl-5-heptenal, (+/−)-3/4-(4-hydroxy-4-methylpentyl)-3-cyclohexene-1-carbaldehyde, (+/−)-3-(4-methoxyphenyl)-2-methylpropanal, (+/−)-8alpha/9alpha-methoxy-1alpha,2beta, 6beta-tricyclo[5.2.1.0^{2,6}]decane-3beta-carbaldehyde, 3,5,6-trimethyl-3-cyclohexene-1-carbaldehyde, 2,4,6-trimethyl-3-cyclohexene-1-carbaldehyde, (+/−)-2-methyldecanal, (+/−)-2,6,10-trimethyl-9-undecenal, (1RS,6RS)-3,6-dimethyl-3-cyclohexene-1-carbaldehyde, (1RS,6RS)-4,6-dimethyl-3-cyclohexene-1-carbaldehyde, (1RS,6SR)-4,6-dimethyl-3-cyclohexene-1-carbaldehyde, (+/−)-8,8-dimethyl-1,2,3,4,5,6,7,8-octahydro-2-naphthalenecarbaldehyde, (+/−)-5,5-dimethyl-1,2,3,4,5,6,7,8-octahydro-2-naphthalenecarbaldehyde, (+/−)-2,2-dimethyltricyclo[6.2.1.0^{1,6}]undecan-7-one, 6-methoxy-2,6-dimethyloctanal, 7-isopropyl-5-methylbicyclo[2.2.2]oct-5-ene-2-carbaldehyde, 8-isopropyl-6-methylbicyclo[2.2.2]oct-5-ene-2-carbaldehyde, 3-(4-isobutylphenyl)-2-methylpropanal, octahydro-1H-4,7-methanoindene-2-carbaldehyde, (E)-2,6,10-trimethylundeca-5,9-dienal and any mixture thereof.

In a preferred embodiment, the at least one aldehydic perfumery raw material is selected from the group consisting of 3-(1, 1/3,3-dimethyl-2,3-dihydro-1H-inden-5-yl) propanal, decenal, dodecanal, undecanal, octanal, (+/−)-7-hydroxy-3,7-dimethyloctanal, 10-undecenal, nonanal, 10-undecenal, (9E)-9-undecenal, (9Z)-9-undecenal, 3-(1,1/3,3-dimethyl-2,3-dihydro-1H-inden-5-yl) propanal, (+/−)-3-(3-isopropyl-1-phenyl) butanal, 3-(4-tert-butylphenyl) propanal, (+)—(R)-3,7-dimethyl-6-octenal, (+/−)-3,7-dimethyl-6-octenal, (+/−)-3-phenylbutanal, 3-[(1R)-4-methyl-3-cyclohexen-1-yl]butanal, (4E)-4-methyl-5-(4-methylphenyl)-4-pentenal, 3-(4,4-dimethyl-1-cyclohexen-1-yl) propanal, phenylacetaldehyde, hexanal, (E)-4-decenal, 7-hydroxy-3,7-dimethyloctanal, (4Z)-4-dodecenal, tridecanal, heptanal, 3-phenylpropanal, 3-methylbutanal, (4-methylphenyl) acetaldehyde, (E)-3,7,11-trimethyldodeca-6,10-dienal, 4-(octahydro-5H-4,7-methanoinden-5-ylidene) butanal, 3-(4-isopropylphenyl) propanal, 4,8-dimethyldeca-4,9-dienal, (E)-5,9-dimethyldeca-4,8-dienal, 2-((3,7-dimethylocta-2,6-dien-1-yl)oxy) acetaldehyde, (4Z,7Z)-deca-4,7-dienal, (S,Z)-6-(2,2,3-trimethylcyclopent-3-en-1-yl) hex-4-enal, 6,8-dimethylnon-7-enal and any mixture thereof.

In a particular embodiment, the at least one aldehydic perfumery raw material is 3-(1,1/3,3-dimethyl-2,3-dihydro-1H-inden-5-yl) propanal. In another embodiment, the at least one aldehydic perfumery raw material is decenal. In another embodiment, the at least one aldehydic perfumery raw material is dodecanal. In another embodiment, the at least one aldehydic perfumery raw material is undecanal. In another embodiment, the at least one aldehydic perfumery raw material is octanal. In another embodiment, the at least one aldehydic perfumery raw material is (+/−)-7-hydroxy-3,7-dimethyloctanal. In another embodiment, the at least one aldehydic perfumery raw material is 10-undecenal. In another embodiment, the at least one aldehydic perfumery raw material is (9E)-9-undecenal. In another embodiment, the at least one aldehydic perfumery raw material is (9Z)-9-undecenal. In another embodiment, the at least one aldehydic perfumery raw material is 3-(1, 1/3,3-dimethyl-2,3-dihydro-1H-inden-5-yl) propanal. In another embodiment, the at least one aldehydic perfumery raw material is (+/−)-3-(3-isopropyl-1-phenyl) butanal. In another embodiment, the at least one aldehydic perfumery raw material is (+)—(R)-3,7-dimethyl-6-octenal. In another embodiment, the at least one aldehydic perfumery raw material is (+/−)-3,7-dimethyl-6-octenal. In another embodiment, the at least one aldehydic perfumery raw material is (+/−)-3-phenylbutanal. In another embodiment, the at least one aldehydic perfumery raw material is 3-[(1R)-4-methyl-3-cyclohexen-1-yl]butanal. In another embodiment, the at least one aldehydic perfumery raw material is (4E)-4-methyl-5-(4-methylphenyl)-4-pentenal. In another embodiment, the at least one aldehydic perfumery raw material is 3-(4,4-dimethyl-1-cyclohexen-1-yl) propanal. In another embodiment, the at least one aldehydic perfumery raw material is phenylacetaldehyde. In another embodiment, the at least one aldehydic perfumery raw material is hexanal. In another embodiment, the at least one aldehydic perfumery raw material is (E)-4-decenal. In another embodiment, the at least one aldehydic perfumery raw material is 7-hydroxy-3,7-dimethyloctanal. In another embodiment, the at least one aldehydic perfumery raw material is (4Z)-4-dodecenal. In another embodiment, the at least one aldehydic perfumery raw material is tridecanal. In another embodiment, the at least one aldehydic perfumery raw material is heptanal. In another embodiment, the at least one aldehydic perfumery raw material is 3-phenylpropanal. In another embodiment, the at least one aldehydic perfumery raw material is 3-methylbutanal. In another embodiment, the at least one aldehydic perfumery raw material is (4-methylphenyl) acetaldehyde.

Amounts of the at least one aldehydic perfumery raw material in the perfume composition that are effective in reducing the formation of hydrogen sulfide from the sulfur-containing pro-fragrance can be determined by a skilled person.

For example, a certain amount of the at least one aldehydic perfumery raw material can be combined with the at least one sulfur-containing pro-fragrance and optionally with other perfumery raw materials. The mixture can then be heated at a temperature of 50° C. for two weeks. Subsequent sensorial evaluation may allow the conclusion whether the addition of a certain amount of aldehydic perfumery raw material was effective in reducing the smell of hydrogen sulfide compared to a control sample that did not comprise the at least one aldehydic perfumery raw material. Alternatively, the release of hydrogen sulfide may be determined upon two weeks of a storage at 50° C. via chromatography-mass spectrometry (GC-MS), optionally combined with in solid-phase microextraction (SPME), and compared with a control sample.

Hence, in a particular embodiment, an effective amount of aldehydic perfumery raw material for reducing the formation of hydrogen sulfide is determined by measuring the release of hydrogen sulfide upon storage of the perfume composition at 50° C. for two weeks. The release of hydrogen sulfide is preferably measured via sensorial evaluation and/or chromatography-mass spectrometry (GC-MS), optionally combined with solid-phase microextraction (SPME).

In a particular embodiment, the perfume composition comprises the at least one aldehydic perfumery raw material in an amount of at least 0.75 wt. %, preferably of at least 1.5 wt. %, more preferably of at least 2.5 wt. %, most preferably of at least 5 wt. %, based on the total weight of the perfume composition. In said embodiment, the at least one aldehydic perfumery raw material preferably shows a chemical structure of R—CH₂—CHO.

In a particular embodiment, the perfume composition comprises the at least one aldehydic perfumery raw material in an amount of at least 2.5 wt. %, based on the total weight of the perfume composition. In said embodiment, the at least one aldehydic perfumery raw material preferably shows a chemical structure of R′—CH(R′)—CHO as defined above.

In a particular embodiment, the perfume composition comprises the at least one aldehydic perfumery raw material in an amount of at most 85 wt. %, preferably at most 80 wt. %, preferably at most 70 wt. %, preferably at most 60 wt. %, preferably at most 50 wt. %, preferably at most 40 wt. %, preferably at most 20 wt. %, even more preferably at most 20 wt. %. In said embodiment, the at least one aldehydic perfumery raw material preferably shows a chemical structure of R—CH₂—CHO as defined above.

In a particular embodiment, the perfume composition comprises the at least one aldehydic perfumery raw material in an amount of from 0.75 to 85 wt. %, preferably from 0.75 to 80 wt. %, preferably from 0.75 to 70 wt. %, preferably from 0.75 to 60 wt. %, preferably from 0.75 to 50 wt. %, preferably from 0.75 to 40 wt. %, preferably from 0.75 to 20 wt. %, even more preferably from 0.75 to 20 wt. %. In said embodiment, the at least one aldehydic perfumery raw material preferably shows a chemical structure of R—CH₂—CHO as defined above.

In a particular embodiment, the perfume composition comprises the at least one aldehydic perfumery raw material in an amount of from 1 to 85 wt. %, preferably from 1 to 80 wt. %, preferably from 1 to 70 wt. %, preferably from 1 to 60 wt. %, preferably from 1 to 50 wt. %, preferably from 1 to 40 wt. %, preferably from 1 to 20 wt. %, even more preferably from 1 to 20 wt. %. In said embodiment, the at least one aldehydic perfumery raw material preferably shows a chemical structure of R—CH₂—CHO as defined above.

In a particular embodiment, the perfume composition comprises the at least one aldehydic perfumery raw material in an amount of from 1.5 to 85 wt. %, preferably from 1.5 to 80 wt. %, preferably from 1.5 to 70 wt. %, preferably from 1.5 to 60 wt. %, preferably from 1.5 to 50 wt. %, preferably from 1.5 to 40 wt. %, preferably from 1.5 to 20 wt. %, even more preferably from 1.5 to 20 wt. %. In said embodiment, the at least one aldehydic perfumery raw material preferably shows a chemical structure of R—CH₂—CHO as defined above.

In a particular embodiment, the perfume composition comprises the at least one aldehydic perfumery raw material in an amount of from 2.5 to 85 wt. %, preferably from 2.5 to 80 wt. %, preferably from 2.5 to 70 wt. %, preferably from 2.5 to 60 wt. %, preferably from 2.5 to 50 wt. %, preferably from 2.5 to 40 wt. %, preferably from 2.5 to 20 wt. %, even more preferably from 2.5 to 20 wt. %. In said embodiment, the at least one aldehydic perfumery raw material preferably shows a chemical structure of R—CH₂—CHO as defined above.

In a particular embodiment, the perfume composition comprises the at least one aldehydic perfumery raw material in an amount of from 0.75 to 5 wt. %.

Apart from the aldehydic perfumery raw materials mentioned above, the perfume composition according to the present invention may further comprise non-aldehydic perfumery raw materials and other aldehydic perfumery raw materials.

Various perfumery raw materials are well known to a skilled person in the art and their nature does not warrant a detailed description here, which in any case would not be exhaustive, the skilled perfumer being able to select them on the basis of his general knowledge and according to the intended use or application and the organoleptic effect it is desired to achieve. In general terms, these perfumery raw materials belong to chemical classes as varied as alcohols, lactones, aldehydes, ketones, esters, ethers, acetates, nitriles, thiols, terpenoids, nitrogenous or sulphureous heterocyclic compounds and essential oils, and perfumery raw materials can be of natural or synthetic origin. Many of these perfuming ingredients are listed in reference texts such as in the book by S. Arctander, Perfume and Flavor Chemicals, 1969, Montclair, N.J., USA, or its more recent versions, or in other works of similar nature such as Fenaroli's Handbook of Flavor Ingredients, 1975, CRC Press or Synthetic Food Adjuncts, 1947, by M. B. Jacobs, Van Nostrand Co., Inc. Suitable solvents and adjuvants are also well known in the art.

Perfumery raw materials (including aldehydic and non-aldehydic perfumery raw materials) may be, for example:

Aldehydic ingredients: (2E)-2-benzylideneoctanal, 4-methoxybenzaldehyde, 3-ethoxy-4-hydroxybenzaldehyde, 3,7-dimethyl-2,6-octadienal, (E)-2-pentyl-3-phenyl-2-propenal, 4-hydroxy-3-methoxybenzaldehyde, benzo[d][1,3]dioxole-5-carbaldehyde, benzaldehyde, (E)-3-phenyl-2-propenal, 3-(2/4-ethylphenyl)-2,2-dimethylpropanal, (2E)-2-methyl-3-phenyl-2-propenal, 3,7-dimethyl-2,6-octadienal, (2E)-2-benzylideneoctanal, (+/−)-1-methyl-4-(4-methyl-3-pentenyl)-3-cyclohexene-1-carbaldehyde, (2E)-2-dodecenal, (2E,6Z)-2,6-nonadienal, 4-(2-propanyl)benzaldehyde, 4-methylbenzaldehyde, 4-formyl-2-methoxyphenyl 2-methylpropanoate, (3,4-dimethoxybenzylidene) (methyl)-λ3-oxidane, (2E,6Z)-2,6-nonadienal, (E)-2-decenal, (E)-2-tridecenal, 3,6,7-trimethyl-2,6-octadienal, and 2-hydroxybenzaldehyde;

Aromatic-herbal ingredients: eucalyptus oil, camphor, eucalyptol, 5-methyltricyclo[6.2.1.0˜2,7˜]undecan-4-one, 1-methoxy-3-hexanethiol, 2-ethyl-4,4-dimethyl-1,3-oxathiane, 2,2,7/8,9/10-Tetramethylspiro[5.5]undec-8-en-1-one, menthol and/or alpha-pinene;

Balsamic ingredients: coumarin, ethylvanillin and/or vanillin;

Citrus ingredients: dihydromyrcenol, citral, orange oil, linalyl acetate, citronellyl nitrile, orange terpenes, limonene, 1-p-menthen-8-yl acetate and/or 1,4 (8)-p-menthadiene; Floral ingredients: methyl dihydrojasmonate, linalool, citronellol, phenylethanol, 3-(4-tert-butylphenyl)-2-methylpropanal, hexylcinnamic aldehyde, benzyl acetate, benzyl salicylate, tetrahydro-2-isobutyl-4-methyl-4 (2H)-pyranol, beta ionone, methyl 2-(methylamino)benzoate, (E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, 1-(2,6,6-trimethyl-1,3-cyclohexadien-1-yl)-2-buten-1-one, (2E)-1-(2,6,6-trimethyl-2-cyclohexen-1-yl)-2-buten-1-one, (2E)-1-[2,6,6-trimethyl-3-cyclohexen-1-yl]-2-buten-1-one, (2E)-1-(2,6,6-trimethyl-1-cyclohexen-1-yl)-2-buten-1-one, 2,5-dimethyl-2-indanmethanol, 2,6,6-trimethyl-3-cyclohexene-1-carboxylate, 3-(4,4-dimethyl-1-cyclohexen-1-yl) propanal, hexyl salicylate, 3,7-dimethyl-1,6-nonadien-3-ol, 3-(4-isopropylphenyl)-2-methylpropanal, verdyl acetate, geraniol, p-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, verdyl proprionate, geranyl acetate, tetrahydro linalool, cis-7-p-menthanol, propyl(S)-2-(1,1-dimethylpropoxy) propanoate, 2-methoxynaphthalene, 2,2,2-trichloro-1-phenylethyl acetate, 4/3-(4-hydroxy-4-methylpentyl)-3-cyclohexene-1-carbaldehyde, amylcinnamic aldehyde, 8-decen-5-olide, 4-phenyl-2-butanone, isononyle acetate, 4-(1,1-dimethylethyl)-1-cyclohexyl acetate, verdyl isobutyrate and/or mixture of methylionones isomers;

Fruity ingredients: gamma-undecalactone, 2,2,5-trimethyl-5-pentylcyclopentanone, 2-methyl-4-propyl-1,3-oxathiane, 4-decanolide, ethyl 2-methyl-pentanoate, hexyl acetate, ethyl 2-methylbutanoate, gamma-nonalactone, allyl heptanoate, 2-phenoxyethyl isobutyrate, ethyl 2-methyl-1,3-dioxolane-2-acetate, 3-(3,3/1,1-dimethyl-5-indanyl) propanal, diethyl 1,4-cyclohexanedicarboxylate, 3-methyl-2-hexen-1-yl acetate, 1-[3,3-dimethylcyclohexyl]ethyl[3-ethyl-2-oxiranyl]acetate and/or diethyl 1,4-cyclohexane dicarboxylate;

Green ingredients: 2-methyl-3-hexanone (E)-oxime, 2,4-dimethyl-3-cyclohexene-1-carbaldehyde, 2-tert-butyl-1-cyclohexyl acetate, styrallyl acetate, allyl(2-methylbutoxy)acetate, 4-methyl-3-decen-5-ol, diphenyl ether, (Z)-3-hexen-1-ol and/or 1-(5,5-dimethyl-1-cyclohexen-1-yl)-4-penten-1-one;

Musk ingredients: 1,4-dioxa-5,17-cycloheptadecanedione, (Z)-4-cyclopentadecen-1-one, 3-methylcyclopentadecanone, 1-oxa-12-cyclohexadecen-2-one, 1-oxa-13-cyclohexadecen-2-one, (9Z)-9-cycloheptadecen-1-one, 2-{1S)-1-[(1R)-3,3-dimethylcyclohexyl]ethoxy}-2-oxoethyl propionate 3-methyl-5-cyclopentadecen-1-one, 1,3,4,6,7,8-hexahydro-4,6,6,7,8,8-hexamethyl-cyclopenta-g-2-benzopyrane, (1S,1′R)-2-[1-(3′,3′-dimethyl-1′-cyclohexyl) ethoxy]-2-methylpropyl propanoate, oxacyclohexadecan-2-oneand/or (1S, 1′R)-[1-(3′,3′-dimethyl-1′-cyclohexyl) ethoxycarbonyl]methyl propanoate; Woody ingredients: 1-[(1RS,6SR)-2,2,6-trimethylcyclohexyl]-3-hexanol, 3,3-dimethyl-5-[(1R)-2,2,3-trimethyl-3-cyclopenten-1-yl]-4-penten-2-ol, 3,4′-dimethylspiro[oxirane-2,9′-tricyclo[6.2.1.02,7]undec[4]ene, (1-ethoxyethoxy)cyclododecane, 2,2,9,11-tetramethylspiro[5.5]undec-8-en-1-yl acetate, 1-(octahydro-2,3,8,8-tetramethyl-2-naphtalenyl)-1-ethanone, patchouli oil, terpenes fractions of patchouli oil, Clearwood®, (1′R, E)-2-ethyl-4-(2′,2′,3′-trimethyl-3′-cyclopenten-1′-yl)-2-buten-1-ol, 2-ethyl-4-(2,2,3-trimethyl-3-cyclopenten-1-yl)-2-buten-1-ol, methyl cedryl ketone, 5-(2,2,3-trimethyl-3-cyclopentenyl)-3-methylpentan-2-ol, 1-(2,3,8,8-tetramethyl-1,2,3,4,6,7,8,8a-octahydronaphthalen-2-yl) ethan-1-one and/or isobornyl acetate;

Other ingredients (e.g. amber, powdery spicy or watery): dodecahydro-3a,6,6,9a-tetramethyl-naphtho [2,1-b]furan and any of its stereoisomers, heliotropin, anisic aldehyde, eugenol, cinnamic aldehyde, clove oil, 3-(1,3-benzodioxol-5-yl)-2-methylpropanal, 7-methyl-2H-1,5-benzodioxepin-3 (4H)-one, 2,5,5-trimethyl-1,2,3,4,4a,5,6,7-octahydro-2-naphthalenol, 1-phenylvinyl acetate, 6-methyl-7-oxa-1-thia-4-azaspiro[4.4]nonan and/or 3-(3-isopropyl-1-phenyl) butanal.

In a particular embodiment, the perfume composition comprises perfumery raw materials in an amount of from 10 to 99 wt. %, preferably from 10 to 95 wt. %, more preferably from 20 to 90 wt. %, even more preferably from 30 to 80 wt. %, based on the total weight of the perfume composition. This includes all kinds of perfumery raw materials.

In a particular embodiment, the perfume composition consists of perfumery raw materials and the at least one sulfur-containing pro-fragrance compound.

In a particular embodiment, the weight ratio of perfumery raw materials to the at least one sulfur-containing pro-fragrance compound in the perfume composition is from 1000:1 to 1:10, preferably, from 100:1 to 1:1, more preferably from 50:1 to 4:1.

In a particular embodiment, the perfume composition according to the invention comprises a hydrogen sulfide scavenger.

Under “hydrogen sulfide scavenger”, a compound is meant that is capable of trapping hydrogen sulfide with the consequence that the unpleasant odor of hydrogen sulfide is reduced or cannot be perceived any more.

In a particular embodiment, the hydrogen sulfide scavenger is an amine selected from the group consisting of monoethanolamine, diethanolamine, triethanolamine, methyldiethanolamine, dimethylethanolamine, alkyldiethanolamines, ethoxylated alkyldiethanolamines, 1,1′,1″,1″-[1,2-ethanediyldi (nitrilo)]tetra(2-propanol), and any mixture thereof. Preferably, the hydrogen sulfide scavenger is a tertiary amine selected from the group consisting of triethanolamine, methyldiethanolamine, dimethylethanolamine, alkyldiethanolamines, ethoxylated alkyldiethanolamines, 1,1′,1″,1″-[1,2-ethanediyldi (nitrilo)]tetra(2-propanol), and any mixture thereof.

In a particular embodiment, the perfume composition comprises the hydrogen sulfide scavenger in an amount of from 0.1 to 10 wt. %, preferably from 0.5 to 5 wt. %.

In a particular embodiment, the ratio of hydrogen sulfide scavenger to sulfur-containing pro-fragrance compound is from 2:1 to 1:20, preferably from 1:1 to 1:10.

In a particular embodiment, the perfume composition is fully or partly encapsulated. The perfume composition can be encapsulated in a microcapsule. Preferably, the perfume composition is encapsulated in a core-shell microcapsule wherein the perfume composition is contained in the core surrounded by the shell. The shell of the microcapsule protects the perfume composition from the environment. The shell is made of material, which is able to release the perfume composition. Preferably, the shell is made of material, which is able to release the perfume composition upon breakage of the shell and/or by diffusion through the shell. A person skilled in the art is well aware of processes to prepare said microcapsules.

The nature of the shell can vary. According to a particular embodiment, the shell of the microcapsule comprises a material selected from the group consisting of polyurea, polyurethane, polyamide, polyester, poly(meth)acrylate (i.e. polyacrylate and/or polymethacrylate), polysiloxane, polycarbonate, polysulfonamide, polymers of urea and formaldehyde, melamine and formaldehyde, melamine and urea, or melamine and glyoxal and mixtures thereof. The shell can also be hybrid, namely organic-inorganic such as a hybrid shell composed of at least two types of inorganic particles that are cross-linked, or yet a shell resulting from the hydrolysis and condensation reaction of a polyalkoxysilane macro-monomeric composition.

According to a particular embodiment, the core-shell microcapsule(s) can be also prepared by using different encapsulation methods.

In a preferred embodiment, the shell of the microcapsules may be, each independently, selected from the group of aminoplast, polyamide, polyester, polyurea and polyurethane shells and mixtures thereof.

In a particular embodiment, the shell of the microcapsules comprises an aminoplast copolymer, such as melamine-formaldehyde or urea-formaldehyde or cross-linked melamine formaldehyde or melamine glyoxal.

In a particular embodiment, the shell of the microcapsules is polyurea-based made from, for example but not limited to isocyanate-based monomers and amine-containing crosslinkers such as guanidine carbonate and/or guanazole. Certain polyurea microcapsules comprise a polyurea wall which is the reaction product of the polymerisation between at least one polyisocyanate comprising at least two isocyanate functional groups and at least one reactant selected from the group consisting of an amine (for example a water-soluble guanidine salt and guanidine); a colloidal stabilizer or emulsifier; and an encapsulated perfume. However, the use of an amine can be omitted.

In a particular embodiment, the shell of the microcapsules is polyurethane-based made from, for example but not limited to polyisocyanate and polyols, polyamide, polyester, etc.

In a particular embodiment, the microcapsules have a polymeric shell resulting from complex coacervation wherein the shell is possibly cross-linked.

In a particular embodiment, the coacervate comprises a first polyelectrolyte, preferably selected among proteins (such as gelatin), polypeptides or polysaccharides (such as chitosan), most preferably Gelatin and a second polyelectrolyte, preferably alginate salts, cellulose derivatives guar gum, pectinate salts, carrageenan, polyacrylic and methacrylic acid or xanthan gum, or yet plant gums such as acacia gum (Gum Arabic), most preferably Gum Arabic.

The coacervate first material can be hardened chemically using a suitable cross-linker such as glutaraldehyde, glyoxal, formaldehyde, tannic acid or genipin or can be hardened enzymatically using an enzyme such as transglutaminase.

The second polymeric material can be selected from the group consisting of polyurea, polyurethane, polyamide, polyester, polyacrylate, polysiloxane, polycarbonate, polysulfonamide, polymers of urea and formaldehyde, melamine and formaldehyde, melamine and urea, or melamine and glyoxal and mixtures thereof, preferably polyurea and/or polyurethane.

In a particular embodiment, the perfume composition according to the invention comprises a perfumery carrier.

By “perfumery carrier” it is meant here a material which is practically neutral from a perfumery point of view, i.e. that does not significantly alter the organoleptic properties of perfuming ingredients. Said carrier may be a liquid or a solid.

As liquid carrier one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting examples, solvents such as butylene or propylene glycol, glycerol, dipropyleneglycol and its monoether, 1,2,3-propanetriyl triacetate, dimethyl glutarate, dimethyl adipate 1,3-diacetyloxypropan-2-yl acetate, diethyl phthalate, isopropyl myristate, benzyl benzoate, benzyl alcohol, 2-(2-ethoxyethoxy)-1-ethano, tri-ethyl citrate or mixtures thereof, which are the most commonly used. Other suitable perfumery carriers than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company), or hydrogenated castors oils such as those known under the trademark Cremophor® RH 40 (origin: BASF).

Solid carrier is meant to designate a material to which the perfumed composition or some element of the perfumed composition can be chemically or physically bound. In general, such solid carriers are employed either to stabilize the composition, or to control the rate of evaporation of the compositions or of some ingredients. The use of solid carrier is of current use in the art and a person skilled in the art knows how to reach the desired effect. However, by way of non-limiting example of solid carriers, one may cite absorbing gums or polymers or inorganic material, such as porous polymers, cyclodextrins, wood based materials, organic or inorganic gels, clays, gypsum talc or zeolites.

As other non-limiting examples of solid carriers, one may cite encapsulating materials. Examples of such materials may comprise wall-forming and plasticizing materials, such as mono, di- or trisaccharides, natural or modified starches, hydrocolloids, cellulose derivatives, polyvinyl acetates, polyvinylalcohols, proteins or pectins, or yet the materials cited in reference texts such as H. Scherz, Hydrokolloide: Stabilisatoren, Dickungs-und Geliermittel in Lebensmitteln, Band 2 der Schriftenreihe Lebensmittelchemie, Lebensmittelqualität, Behr's Verlag Gmbh & Co., Hamburg, 1996. The encapsulation is a well-known process to a person skilled in the art, and may be performed, for instance, by using techniques such as spray-drying, agglomeration or yet extrusion; or consists of a coating encapsulation, including coacervation and complex coacervation technique.

As non-limiting examples of solid carriers, one may cite in particular the core-shell capsules with resins of aminoplast, polyamide, polyester, polyurea or polyurethane type or a mixture thereof (all of said resins are well known to a person skilled in the art) using techniques like phase separation process induced by polymerization, interfacial polymerization, coacervation or altogether (all of said techniques have been described in the prior art), optionally in the presence of a polymeric stabilizer or of a cationic copolymer.

Resins may be produced by the polycondensation of an aldehyde (e.g. formaldehyde, 2,2-dimethoxyethanal, glyoxal, glyoxylic acid or glycolaldehyde and mixtures thereof) with an amine such as urea, benzoguanamine, glycoluryl, melamine, methylol melamine, methylated methylol melamine, guanazole and the like, as well as mixtures thereof. Alternatively, one may use preformed resins alkylolated polyamines such as those commercially available under the trademark Urac® (origin: Cytec Technology Corp.), Cymel® (origin: Cytec Technology Corp.), Urecoll® or Luracoll® (origin: BASF).

Others resins one are the ones produced by the polycondensation of an a polyol, like glycerol, and a polyisocyanate, like a trimer of hexamethylene diisocyanate, a trimer of isophorone diisocyanate or xylene diisocyanate or a Biuret of hexamethylene diisocyanate or a trimer of xylene diisocyanate with trimethylolpropane (known with the tradename of Takenate®, origin: Mitsui Chemicals), among which a trimer of xylene diisocyanate with trimethylolpropane and a Biuret of hexamethylene diisocyanate.

In a particular embodiment, the perfume composition according to the invention comprises at least one perfumery adjuvant.

The term “perfumery adjuvant” is understood as an ingredient capable of imparting additional added benefit such as a color, a particular light resistance, chemical stability and etc. A detailed description of the nature and type of adjuvant commonly used in perfuming bases cannot be exhaustive, but it has to be mentioned that the ingredients are well known to a person skilled in the art. However, one may cite as specific non-limiting examples the following: viscosity agents (e.g. surfactants, thickeners, gelling and/or rheology modifiers), stabilizing agents (e.g. preservatives, antioxidants, heat/light and or buffers or chelating agents, such as BHT), coloring agents (e.g. dyes and/or pigments), preservatives (e.g. antibacterial or antimicrobial or antifungal or anti-irritant agents), abrasives, skin cooling agents, fixatives, insect repellants, ointments, vitamins and mixture thereof. By “fixative” also called “modulator”, it is understood here an agent having the capacity to affect the manner in which the odour, and in particular the evaporation rate and intensity, of the compositions incorporating said modulator can be perceived by an observer or user thereof, over time, as compared to the same perception in the absence of the modulator. In particular, the modulator allows prolonging the time during which their fragrance is perceived. Non-limiting examples of suitable modulators may include methyl glucoside polyol; ethyl glucoside polyol; propyl glucoside polyol; isocetyl alcohol; PPG-3 myristyl ether; neopentyl glycol diethylhexanoate; sucrose laurate; sucrose dilaurate, sucrose myristate, sucrose palmitate, sucrose stearate, sucrose distearate, sucrose tristearate, hyaluronic acid disaccharide sodium salt, sodium hyaluronate, propylene glycol propyl ether; dicetyl ether; polyglycerin-4 ethers; isoceteth-5; isoceteth-7, isoceteth-10; isoceteth-12; isoceteth-15; isoceteth-20; isoceteth-25; isoceteth-30; disodium lauroamphodipropionate; hexaethylene glycol monododecyl ether; and their mixtures; neopentyl glycol diisononanoate; cetearyl ethylhexanoate; panthenol ethyl ether, DL-panthenol, N-hexadecyl n-nonanoate, noctadecyl n-nonanoate, a profragrance, cyclodextrin, an encapsulation, and a combination thereof. At most 20% by weight, based on the total weight of the perfuming composition, of the modulator may be incorporated into the perfumed consumer product.

In a particular embodiment, the perfume composition comprises at least one compound selected amongst the isothiazolones of formula

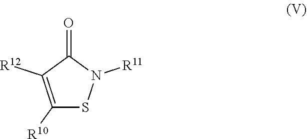

wherein
R¹² and R¹⁰ represent, separately and independently of each other, a hydrogen atom, a halogen atom, preferably chlorine, a C₁-C₄ linear or branched alkyl group, an amino group or a benzylamino group; or, alternatively, R¹² and R¹⁰ are taken together to represent a phenyl or pyridine ring, possibly substituted with one to four C₁-C₄ linear or branched alkyl or alkenyl groups and/or one to two halogen atoms, preferably chlorine atoms; and R¹¹ represents a hydrogen atom, an alkali metal atom, in particular Na or K, a phenyl or benzyl group possibly substituted with one or two halogen atoms and/or one or two methyl, trifluoromethyl, methoxy or amino groups, an amine group, or a C₁-C₈ unsaturated, linear, branched or cyclic hydrocarbon group possibly substituted with one or two nitrogen, oxygen or halogen atoms.

According to a particular embodiment of the invention said compound of formula (V) is one wherein R¹² and R¹⁰ represent, separately and independently of each other, a hydrogen atom, a chlorine atom or a methyl group or, alternatively, R¹² and R¹⁰ are taken together to represent a phenyl ring, and R¹¹ represents a hydrogen atom or a methyl group.

According to a particular embodiment of the invention, said compound of formula (V) is selected from the group of isothiazolones consisting of 1,2-benzisothiazol-3 (2H)-one, 4- or 5-chloro-2-methylisothiazol-3 (2H)-one or 2-methylisothiazol-3 (2H)-one, or more preferably 5-chloro-2-methylisothiazol-3 (2H)-one 1,2-benzisothiazol-3 (2H)-one, and most preferably 1,2-benzisothiazol-3 (2H)-one.

According to a particular embodiment of the invention, said compound of formula (V) is present in the perfume composition of the invention at a weight concentration of 0.0% to 5%, based on the total weight of the perfume composition. According to more preferred embodiments of the invention, the concentration of compound of formula (V) is comprised between 0.001 and 3% of the total weight, preferably between 0.005 and 0.1%.

In a particular embodiment, the perfume composition according to the invention does not comprise more than 19 wt. %, preferably not more than 10 wt. % of perfumery raw materials that promote the formation of hydrogen sulfide from the sulfur-containing pro-fragrance.

Perfumery raw materials promoting the formation of hydrogen sulfide from the sulfur-containing pro-fragrance may induce a hydrogen sulfide smell when being mixed in a 1:1 (w/w) ratio with the sulfur-containing pro-fragrance compound and stored for 2 weeks at 50° C. Preferably, a hydrogen sulfide smell is induced when being mixed in a 1:1 (w/w) ratio with 3-(dodecylthio)-1-(2,6,6-trimethylcyclohex-3-en-1-yl) butan-1-one (HaloScent® D) and stored for 2 weeks at 50° C. Preferably, the mixing is carried out in a 10 mL glass jar.

The following perfumery raw materials may be considered perfumery raw materials that promote the formation of hydrogen sulfide from the sulfur-containing pro-fragrance: 4-(4-hydroxyphenyl)-2-butanone, 2-phenylethanol, (+/−)-2-phenyl-1-propanol, indole, 1-(2-naphthyl) ethanone, (+/−)-alpha terpineol, methyl 2-((1RS,2RS)-3-oxo-2-pentylcyclopentyl)acetate, (+/−)-3,7-dimethyl-1,6-octadien-3-ol, 3,7-dimethyl-2,6-octadien-1-ol, (+/−)-3-methyl-5-phenyl-1-pentanol, (−)—R-3,7-dimethyl-6-octenenitrile, (+/−)-(2,5-dimethyl-2,3-dihydro-1H-inden-2-yl) methanol, (+/−)-2,2,2-trichloro-1-phenylethyl acetate, (+/−)-2,6-dimethyl-7-octen-2-ol, cyclohexylidene(phenyl)acetonitrile, (+/−)-3,7-dimethyl-6-octen-1-ol, (+/−)-3,7-dimethyl-3-octanol, 2-ethoxynaphthalene, (+/−)-1,5-dimethyl-1-vinyl-4-hexenyl acetate, tricyclo[5.2.1.0 (2,6)]dec-3/4-en-8-yl propanoate, (+/−)-2-ethyl-4-(2,2,3-trimethyl-3-cyclopenten-1-yl)-2-buten-1-ol, (−)-(2E)-2-ethyl-4-[(1R)-2,2,3-trimethyl-3-cyclopenten-1-yl]-2-buten-1-ol, allyl 3-cyclohexylpropanoate, (+/−)-1-(2-tert-butyl-1-cyclohexyloxy)-2-butanol, (4E)-3,3-dimethyl-5-[(1R)-2,2,3-trimethyl-3-cyclopenten-1-yl]-4-penten-2-ol, (3Z)-3-hexen-1-yl salicylate, 1-(octahydro-2,3,8,8-tetramethyl-2-naphtalenyl)-1-ethanone, (+)-2-{(1S)-1-[(1R)-3,3-dimethylcyclohexyl]ethoxy}-2-methylpropyl propionate, a mixture of 1-[(1RS,2RS)-1,2,8,8-tetramethyl-1,2,3,4,5,6,7,8-octahydro-2-naphthalenyl]ethanone/1-((2RS,3RS)-2,3,8,8-tetramethyl-1,2,3,4,5,6,7,8-octahydronaphthalen-2-yl) ethanone/1-[(2RS,3RS,8aRS)-2,3,8,8-tetramethyl-1,2,3,5,6,7,8,8a-octahydro-2-naphthalenyl]ethanone/1-[(1RS,2RS,8aSR)-1,2,8,8-tetramethyl-1,2,3,5,6,7,8,8a-octahydro-2-naphthalenyllethanone/1-[(2RS,3RS,8aRS)-2,3,8,8-tetramethyl-1,2,3,4,6,7,8,8a-octahydro-2-naphthalenyl]ethanone, (+/−)-3-endo/exo-methoxy-7,7-dimethyl-10-methylene-bicyclo[4.3.1]decane, (3aRS,5aSR,9aSR,9bRS)-3a,6,6,9a-tetramethyldodecahydronaphtho[2,1-b]furan, 3-methyl-4/5-cyclopentadecen-1-one.

The invention's perfume composition can be advantageously used in all the fields of modern perfumery, i.e. fine or functional perfumery, to positively impart or modify the odor of a consumer product into which said composition is added.

Consequently, another aspect of the invention concerns a consumer product comprising the perfume composition according to the invention.

For the sake of clarity, it is mentioned that, the term “consumer product” is understood as a consumer product, which is expected to deliver at least a pleasant perfuming effect to the surface to which it is applied (e.g. skin, hair, textile, or hard surface). For the sake of clarity, the consumer product is a non-edible product.

The nature and type of the constituents of the perfumed consumer product do not warrant a more detailed description here, which in any case would not be exhaustive, the skilled person being able to select them based on his general knowledge and according to the nature and the desired effect of the product.

Non-limiting examples of suitable consumer products include a perfume, such as a fine perfume, a splash or eau de parfum, a cologne or a shave or after-shave lotion; a fabric care product, such as a liquid, pods or solid detergent or tablets, a fabric softener, a liquid or solid scent booster, a dryer-sheet, a fabric refresher, an ironing water, a paper, a bleach, a carpet cleaner, a curtain-care product; a body-care product, such as a hair care product (e.g. a shampoo, a leave-on or rinse-off hair conditioner, a coloring preparation or a hair spray, a color-care product, a hair shaping product), a dental care product, a disinfectant, an intimate care product; a cosmetic preparation (e.g. a skin cream or lotion, a vanishing cream or a deodorant or antiperspirant (e.g. a spray or roll on), a hair remover, a tanning or sun or after sun product, a nail product, a skin cleansing, a makeup); or a skin-care product (e.g. a soap, a shower or bath mousse, oil or gel, or a hygiene product or a foot/hand care products); an air care product, such as an air freshener or a “ready to use” powdered air freshener which can be used in the home space (rooms, refrigerators, cupboards, shoes or car) and/or in a public space (halls, hotels, malls, etc.,); or a home care product, such as a mold remover, a furniture care product, a wipe, a dish detergent or a hard-surface (e.g. a floor, bath, sanitary or a window-cleaning) cleaner; a leather care product; a car care product, such as a polish, a wax or a plastic cleaner.

Typical examples of fabric detergents or softener compositions into which the perfume composition of the invention can be incorporated are described in WO 97/34986 or in U.S. Pat. Nos. 4,137,180 and 5,236,615 or EP 799 885. Other typical detergent and softening compositions which can be used are described in works such as Ullmann's Encyclopedia of Industrial Chemistry, Vol. 20, Wiley-VCH, Weinheim, p. 355-540 (2012); Flick, Advanced Cleaning Product Formulations, Noye Publication, Park Ridge, New Jersey (1989); Showell, in Surfactant Science Series, vol. 71: Powdered Detergents, Marcel Dekker, New York (1988); Proceedings of the World Conference on Detergents (4th, 1998, Montreux, Switzerland), AOCS print.

In a particular embodiment, the consumer product is a personal care product or home care product, preferably a fabric conditioner, a shower gel or a rinse-off conditioner.

In a particular embodiment, the consumer product has an acidic pH value, preferably a pH below 5.5, preferably from 2.5 to 5.5.

The proportions in which the perfume composition according to the invention can be incorporated into the various aforementioned articles or compositions vary within a wide range of values. These values are dependent upon the nature of the article or product to be perfumed.

In a particular embodiment, the consumer product comprises the perfume composition in an amount of from 0.1 to 10 wt. %, preferably of from 0.2 to 5 wt. %, more preferably from 0.3 to 4 wt. %, even more preferably from 0.4 to 3 wt. % based on the total weight of the consumer product.

In a particular embodiment, the consumer product is a perfume, a fabric care product, a body-care product, a cosmetic preparation, a skin-care product, an air care product or a home care product. Preferably, the consumer product is a fabric softener, a shower gel or a rinse-off hair conditioner.

According to a particular embodiment, the consumer product may also comprise zinc salt such as zinc ricinoleate, zinc acetate and/or zinc stearate, laureth-3, tetrahydroxypropyl ethylenediamine, propylene glycol or a mixture thereof.

According to a particular embodiment of the invention, the invention's consumer product is an all-purpose cleaner comprising an all-purpose cleaner active base in amount comprised between 85 and 100% by weight, based on the total weight of the consumer product.

According to a particular embodiment of the invention, the invention's perfumed consumer product is a liquid fabric softener comprising a fabric softener active base in amount comprised between 85 and 100% by weight, based on the total weight of the perfumed consumer product. The main constituent of the fabric softener active base is water or water-based solvents. The fabric softener active base may comprise dialkyl quaternary ammonium salts, dialkyl ester quaternary ammonium salts, Hamburg esterquat, triethanolamine quat, silicones and mixtures thereof. Optionally, the fabric softener active base may further comprise a viscosity modifier in an amount comprised between 0.05 and 1% by weight, based on the total weight of the liquid base; preferably chosen in the group consisting of calcium chloride.

According to a particular embodiment of the invention, the invention's consumer product is an all-purpose cleaner comprising an all-purpose cleaner active base in amount comprised between 85 and 100% by weight, based on the total weight of the consumer product. The main constituent of the all-purpose cleaner active base is water or water-based solvents. The all-purpose active base may comprise linear alkylbenzene sulfonates (LAS) in an amount comprised between 0 and 4%, preferably 1 and 2%, nonionic surfactant in an amount comprised between 0 and 8%, preferably 2 and 4% and acid such as citric acid in an amount comprised between 0.1 and 0.5%.

According to a particular embodiment of the invention, the invention's perfumed consumer product is shampoo or a shower gel comprising a shampoo or shower gel active base in amount comprised between 85 and 100% by weight, based on the total weight of the perfumed consumer product. The main constituent of the shampoo or shower gel active base is water or water-based solvents. The shampoo or shower gel active base may comprise sodium alkylether sulfate, ammonium alkylether sulfates, alkylamphoacetate, cocamidopropyl betaine, cocamide MEA, alkylglucosides and aminoacid based surfactants.

According to a particular embodiment of the invention, the invention's perfumed consumer product is a soap bar comprising a soap active base in amount comprised between 85 and 100% by weight, based on the total weight of the perfumed consumer product. The soap bar active base may comprise salt of a weak acid, typically, a salt of weak acid, which may be a fatty acid and strong base like sodium hydroxide.

According to a particular embodiment of the invention, the invention's perfumed consumer product is a rinse-off conditioner comprising a rinse-off conditioner active base in amount comprised between 85 and 99.95% by weight, based on the total weight of the perfumed consumer product. The main constituent of the rinse-off conditioner active base is water or water-based solvents. The rinse-off conditioner active base may comprise cetyltrimonium chloride, stearyl trimonium chloride, benzalkonium chloride, behentrimonium chloride and mixture thereof.

According to a particular embodiment of the invention, the invention's consumer product is a liquid detergent comprising liquid detergent active base in amount comprised between 85 and 100% by weight, based on the total weight of the consumer product. The main constituent of the liquid detergent active base is water or water-based solvents. The liquid detergent active base may comprise anionic surfactant such as alkylbenzenesulfonate (ABS), linear alkylbenzene sulfonates (LAS), secondary alkyl sulfonate (SAS), primary alcohol sulfate (PAS), lauryl ether sulfate (LES), sodium lauryl ether sulfate (SLES), methyl ester sulfonate (MES); nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides; ormixtures thereof.

According to a particular embodiment of the invention, the invention's consumer product is a solid detergent comprising a solid detergent active base in amount comprised between 85 and 100% by weight, based on the total weight of the consumer product. The solid detergent active base may comprise at least one surfactant chosen in the group consisting of anionic, nonionic, cationic, zwiterionic surfactant and mixtures thereof. The surfactant in the solid detergent active base is preferably chosen in the group consisting of linear alkene benzene sulphonate (LABS), sodium laureth sulphate, sodium lauryl ether sulphate (SLES), sodium lauryl sulphate (SLS), alpha olefin sulphonate (AOS), methyl ester sulphonates (MES), alkyl polyglyucosides (APG), primary alcohol ethoxylates and in particular lauryl alcohol ethoxylates (LAE), primary alcohol sulphonates (PAS), soap and mixtures thereof. The solid detergent active base may comprise a further component, commonly used in powder detergent consumer product, selected from the group consisting of bleaching agents such as TAED (tetraacetylethylenediamine); buffering agent; builders such as zeolites, sodium carbonate or mixture thereof; soil release or soil suspension polymers; granulated enzyme particles such as cellulase, lipase, protease, mannanase, pectinase or mixtures thereof; corrosion inhibitor; antifoaming; sud suppressing agents; dyes; fillers such as sodium silicate, sodium sulfate or mixture thereof; source of hydrogen peroxide such as sodium percarbonate or sodium perborate; and mixtures thereof.

In a particular embodiment, the consumer product comprises a preservative, preferably selected from the group consisting of benzisothiazolin-3-one, methylchloroisothiazolinone, methylisothiazolinone, and any mixture thereof.

In a particular embodiment, the consumer product consists of the perfume composition according to the invention and a consumer product base. Under a consumer product base, ingredients and components are understood that render the consumer product functional, i.e. ingredients and components are meant that are characteristic for the respective consumer product. Examples for characteristic ingredients are given above for individual consumer products. Hence, the consumer product base may e.g. be a fabric softener base, a shower gel base, or a rinse-off hair conditioner base.

Another aspect of the present invention concerns a method for reducing the formation of hydrogen sulfide from a sulfur-containing pro-fragrance compound comprising the step of adding at least one aldehydic perfumery raw material to the sulfur-containing pro-fragrance compound,
wherein the at least one aldehydic perfumery raw material shows a chemical structure selected from the group consisting of R—CH₂—CHO and R′—CH(R′)—CHO;
wherein R represents a hydrogen atom, or a C₁ to C₁₈ hydrocarbon group, optionally comprising one to three oxygen atoms and/or one to two nitrogen atoms and/or one sulfur atom;
wherein R′ groups, independently from each other, represent or a C₁ to C₁₈ hydrocarbon group, optionally comprising one to three oxygen atoms and/or one to two nitrogen atoms and/or one sulfur atom; or both R′, when taken together, form a C₅₋₁₆ cycloalkyl, C₅₋₁₆ cycloalkenyl, C₄₋₁₄ heterocycloalkyl or C₄₋₁₄ heterocycloalkenyl group, each optionally substituted with one or more of a C₁₋₁₅ alkyl, C₂₋₁₅ alkenyl, C₁₋₁₅ alkoxy, C₃₋₁₅ cycloalkyl, C₅₋₁₅ cycloalkenyl, C₆₋₁₀ aryl and/or C₆₋₁₀ aryloxy group, each optionally substituted with one or more of a C₁₋₈ alkyl, C₁₋₈ alkoxy, carboxylic acid and/or C₁₋₄ carboxylic ester group, wherein the heteroatom represents one or more of an oxygen atom.

In a particular embodiment, the sulfur-containing pro-fragrance compound is present in a perfume composition.

In a particular embodiment, the at least one aldehydic perfumery raw material is added in an amount effective to reduce the formation of hydrogen sulfide from the sulfur-containing pro-fragrance compound.

Another aspect of the present invention concerns the use of at least one aldehydic perfumery raw material for reducing the formation of hydrogen sulfide from a sulfur-containing pro-fragrance, wherein the at least one aldehydic perfumery raw material shows a chemical structure selected from the group consisting of R—CH₂—CHO and R′—CH(R′)—CHO; wherein R represents a hydrogen atom, or a C₁ to C₁₈ hydrocarbon group, optionally comprising one to three oxygen atoms and/or one to two nitrogen atoms and/or one sulfur atom;
wherein R′ groups, independently from each other, represent a C₁ to C₁₈ hydrocarbon group, optionally comprising one to three oxygen atoms and/or one to two nitrogen atoms and/or one sulfur atom; or both R′, when taken together, form a C₅₋₁₆ cycloalkyl, C₅₋₁₆ cycloalkenyl, C₄₋₁₄ heterocycloalkyl or C₄₋₁₄ heterocycloalkenyl group, each optionally substituted with one or more of a C₁₋₁₅ alkyl, C₂₋₁₅ alkenyl, C₁₋₁₅ alkoxy, C₃₋₁₅ cycloalkyl, C₅₋₁₅ cycloalkenyl, C₆₋₁₀ aryl and/or C₆₋₁₀ aryloxy group, each optionally substituted with one or more of a C₁₋₈ alkyl, C₁₋₈ alkoxy, carboxylic acid and/or C₁₋₄ carboxylic ester group, wherein the heteroatom represents one or more of an oxygen atom.

The individual embodiments mentioned above for the perfume composition according to the invention also apply to the method and use according to the invention.

### EXAMPLES

### Example 1

Reducing the formation of hydrogen sulfide from 3-(dodecylthio)-1-(2,6,6-trimethylcyclohex-3-en-1-yl) butan-1-one (HaloScent® D) by the addition of aldehydic perfumery raw materials:

A control perfume composition comprising 3-(dodecylthio)-1-(2,6,6-trimethylcyclohex-3-en-1-yl) butan-1-one (HaloScent® D) at an amount of 15 wt. %, based on total weight of the perfume composition, has been heated in a 15 mL glass jar at a temperature of 50° C. After two and four weeks of heating, the hydrogen sulfide release from the pro-fragrance compound has been assessed by olfactory analysis. Moreover, the hydrogen sulfide release has been determined after 2 weeks of heating by means of chromatography-mass spectrometry (GC-MS) in solid-phase microextraction (SPME) mode.

Further, several perfume composition samples have been prepared by adding various individual aldehydic perfumery raw materials to the control perfume composition at various amounts (wt. %, based on the total weight of the final perfume composition). These perfume composition samples have been tested for their hydrogen sulfide release the same way as described above for the control perfume composition.

The olfactory analysis revealed that there are three different groups of aldehydic perfumery raw materials based on their ability to reduce the formation of hydrogen sulfide from 3-(dodecylthio)-1-(2,6,6-trimethylcyclohex-3-en-1-yl) butan-1-one (HaloScent® D):
Group A (R—CH₂—CHO): very efficient aldehydic perfumery raw materials; said aldehydes already allowed a drastic reduction of perceived hydrogen sulfide already at an amount of 0.75 wt. %.
Group B (R′—CH(R′)—CHO): efficient aldehydic perfumery raw materials; said aldehydes allowed a significant reduction of perceived hydrogen sulfide at an amount of 2.5 wt. %.
Group C (comparative): less efficient aldehydic perfumery raw materials; said aldehydes only allowed for little reduction of perceived hydrogen sulfide even at an amount of 5 wt. % and above.

In Table 1, an overview of the tested aldehydic perfumery raw materials is provided as well as an indication as to which structural group the individual perfumery raw materials belong to. Further, an indication as to the efficiency in reducing the release of hydrogen sulfide (based on their classification in groups A, B, and C, respectively) is given for the individual aldehydic perfumery raw materials.

TABLE 1
Tested aldehydic perfumery raw materials

| | Structural | Efficiency in H₂S |
|---|---|---|
| Aldehydic perfumery raw material | group | reduction (group) |
| 3-(1,1/3,3-dimethyl-2,3-dihydro-1H-inden-5- | R—CH2—CHO | A |
| yl)propanal | | |
| (2E)-2-benzylideneoctanal | R—Ph—CHO | C |
| (+/−)-2-methyl-3-[4-(2-methyl-2- | R′—CH(R′)—CHO | B |
| propanyl)phenyl]propanal | | |
| 4-methoxybenzaldehyde | R—Ph—CHO | C |
| 3-ethoxy-4-hydroxybenzaldehyde | R—Ph—CHO | C |
| (+/−)-2-methylundecanal | R′—CH(R′)—CHO | B |
| decanal | R—CH2—CHO | A |
| 2,4-dimethyl-3-cyclohexene-1-carbaldehyde | R′—CH(R′)—CHO | B |
| (+/−)-3-(4-isopropylphenyl)-2-methylpropanal | R′—CH(R′)—CHO | B |
| 3,7-dimethyl-2,6-octadienal | R—C═CR—CHO | C |
| dodecanal | R—CH2—CHO | A |
| (E)-2-pentyl-3-phenyl-2-propenal | R—C═CR—CHO | C |
| 4-hydroxy-3-methoxybenzaldehyde | R—Ph—CHO | C |
| undecanal | R—CH2—CHO | A |
| octanal | R—CH2—CHO | A |
| benzo[d][1,3]dioxole-5-carbaldehyde | R—Ph—CHO | C |
| (+/−)-3-(1,3-benzodioxol-5-yl)-2-methylpropanal | R′—CH(R′)—CHO | B |
| benzaldehyde | R—Ph—CHO | C |
| (+/−)-3/4-(4-methyl-3-penten-1-yl)-3- | R′—CH(R′)—CHO | B |
| cyclohexene-1-carbaldehyde | | |
| (+/−)-7-hydroxy-3,7-dimethyloctanal | R—CH2—CHO | A |
| (E)-3-phenyl-2-propenal | R—C═CR—CHO | C |
| 10-undecenal | R—CH2—CHO | A |
| nonanal | R—CH2—CHO | A |
| 10-undecenal, (9E)-9-undecenal, and (9Z)-9- | R—CH2—CHO | A |
| undecenal | | |
| (E)-2-dodecenal, (Z)-3-dodecenal, and | mixed | B |
| dodecanal | structures | |
| 3-(2/4-ethylphenyl)-2,2-dimethylpropanal | α-trisubstituted | C |
| | aldehyde | |
| (+/−)-2,6-dimethyl-5-heptenal | R′—CH(R′)—CHO | B |
| (2E)-2-methyl-3-phenyl-2-propenal | R—C═CR—CHO | C |
| 3-(1,1/3,3-dimethyl-2,3-dihydro-1H-inden-5- | R—CH2—CHO | A |
| yl)propanal | | |
| (+/−)-3-(3-isopropyl-1-phenyl)butanal | R—CH2—CHO | A |
| (+/−)-3/4-(4-hydroxy-4-methylpentyl)-3- | R′—CH(R′)—CHO | B |
| cyclohexene-1-carbaldehyde | | |
| (+/−)-3-(4-methoxyphenyl)-2-methylpropanal | R′—CH(R′)—CHO | B |
| 3-(4-tert-butylphenyl)propanal | R—CH2—CHO | A |
| (+)-(R)-3,7-dimethyl-6-octenal | R—CH2—CHO | A |
| 3,7-dimethyl-2,6-octadienal | R—C═CR—CHO | C |
| (+/−)-3,7-dimethyl-6-octenal | R—CH2—CHO | A |
| (+/−)-3-phenylbutanal | R—CH2—CHO | A |
| (+/−)-8alpha/9alpha-methoxy- | R′—CH(R′)—CHO | B |
| 1alpha,2beta,6beta-tricyclo[5.2.1.0^{2,6}]decane- | | |
| 3beta-carbaldehyde | | |
| (2E)-2-benzylideneoctanal | R—C═CR—CHO | C |
| 3,5,6-trimethyl-3-cyclohexene-1-carbaldehyde/ | R′—CH(R′)—CHO | B |
| 2,4,6-trimethyl-3-cyclohexene-1-carbaldehyde | | |
| (+/−)-1-methyl-4-(4-methyl-3-pentenyl)-3- | α-trisubstituted | C |
| cyclohexene-1-carbaldehyde | aldehyde | |
| (+/−)-2-methyldecanal | R′—CH(R′)—CHO | B |
| 3-[(1R)-4-methyl-3-cyclohexen-1-yl]butanal | R—CH2—CHO | A |
| (2E)-2-dodecenal | R—C═CR—CHO | C |
| (2E,6Z)-2,6-nonadienal | R—C═CR—CHO | C |
| (+/−)-2,6,10-trimethyl-9-undecenal | R′—CH(R′)—CHO | B |
| (4E)-4-methyl-5-(4-methylphenyl)-4-pentenal | R—CH2—CHO | A |
| 4-(2-propanyl)benzaldehyde | R—Ph—CHO | C |
| 3-(4,4-dimethyl-1-cyclohexen-1-yl)propanal | R—CH2—CHO | A |
| (1RS,6RS)-3,6-dimethyl-3-cyclohexene-1- | R′—CH(R′)—CHO | B |
| carbaldehyde, (1RS,6RS)-4,6-dimethyl-3- | | |
| cyclohexene-1-carbaldehyde, and (1RS,6SR)- | | |
| 4,6-dimethyl-3-cyclohexene-1-carbaldehyde | | |
| phenylacetaldehyde | R—CH2—CHO | A |
| (+/−)-8,8-dimethyl-1,2,3,4,5,6,7,8-octahydro-2- | R′—CH(R′)—CHO | B |
| naphthalenecarbaldehyde, (+/−)-5,5-dimethyl- | | |
| 1,2,3,4,5,6,7,8-octahydro-2- | | |
| naphthalenecarbaldehyde, and (+/−)-2,2- | | |
| dimethyltricyclo[6.2.1.0^{1,6}]undecan-7-one | | |
| hexanal | R—CH2—CHO | A |
| (E)-4-decenal | R—CH2—CHO | A |
| 4-methylbenzaldehyde | R—C═CR—CHO | C |
| 4-formyl-2-methoxyphenyl2-methylpropanoate | R—Ph—CHO | C |
| 7-hydroxy-3,7-dimethyloctanal | R—CH2—CHO | A |
| (3,4-dimethoxybenzylidene)(methyl)-λ3- | R—Ph—CHO | C |
| oxidane | | |
| (4Z)-4-dodecenal | R—CH2—CHO | A |
| (2E,6Z)-2,6-nonadienal | R—C═CR—CHO | C |
| tridecanal | R—CH2—CHO | A |
| heptanal | R—CH2—CHO | A |
| 3-phenylpropanal | R—CH2—CHO | A |
| (E)-2-decenal | R—C═CR—CHO | C |
| 3-methylbutanal | R—CH2—CHO | A |
| (E)-2-tridecenal | R—C═CR—CHO | C |
| 3,6,7-trimethyl-2,6-octadienal | R—C═CR—CHO | C |
| 2-hydroxybenzaldehyde | Ph—CHO | C |
| (4-methylphenyl)acetaldehyde | R—CH2—CHO | A |

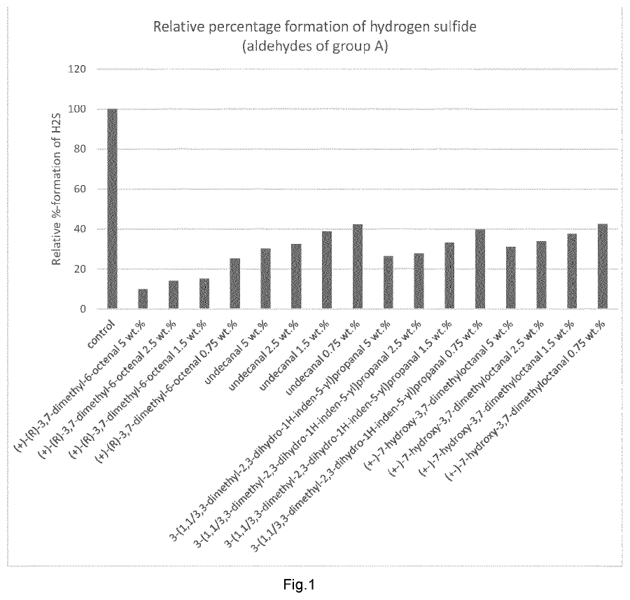

In FIG. 1, the results concerning the release of hydrogen sulfide from 3-(dodecylthio)-1-(2,6,6-trimethylcyclohex-3-en-1-yl) butan-1-one (HaloScent® D) as determined by chromatography-mass spectrometry (GC-MS) are given for several aldehydic perfumery raw materials of group A (very efficient). The release of hydrogen sulfide is expressed relative to the hydrogen sulfide release of the control perfume composition (containing 15 wt. % HaloScent® D and without any aldehydic perfumery raw material added). It can be observed from FIG. 1 that even a concentration of 0.75 wt. % of aldehydic perfumery raw materials of group A already led to a drastic percentage decrease of hydrogen sulfide release compared to the control (without added aldehydic perfumery raw material).

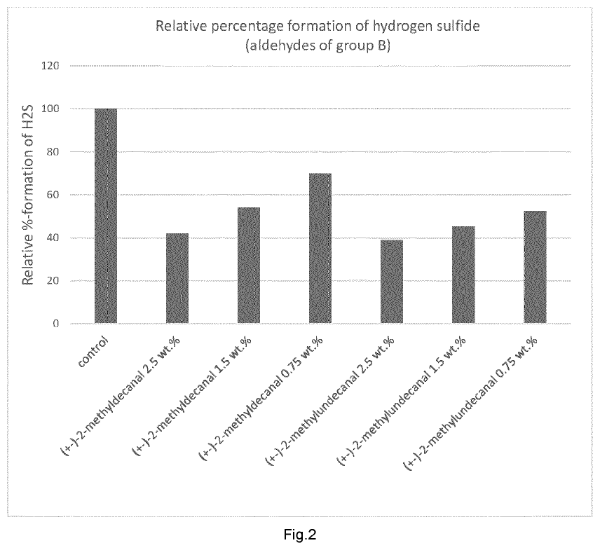

In FIG. 2, the results concerning the release of hydrogen sulfide from 3-(dodecylthio)-1-(2,6,6-trimethylcyclohex-3-en-1-yl) butan-1-one (HaloScent® D) as determined by chromatography-mass spectrometry (GC-MS) are given for several aldehydic perfumery raw materials of group B (efficient). The release of hydrogen sulfide is expressed relative to the hydrogen sulfide release of the control perfume composition (containing 15 wt. % HaloScent® D and without any aldehydic perfumery raw material added). It can be observed from FIG. 2 that at least a concentration of 2.5 wt. % of aldehydic perfumery raw materials of group B led to a drastic percentage decrease of hydrogen sulfide release compared to the control (without added aldehydic perfumery raw material).

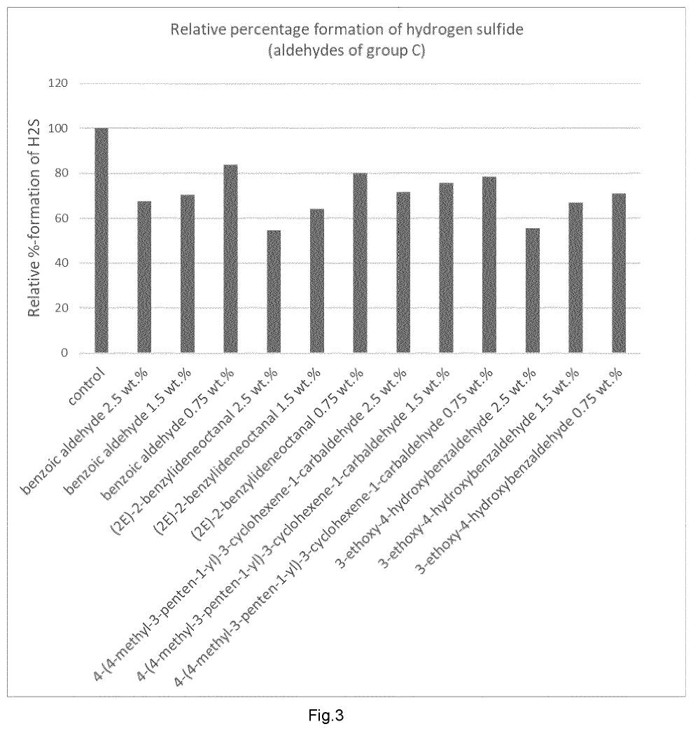

In FIG. 3, the results concerning the release of hydrogen sulfide from 3-(dodecylthio)-1-(2,6,6-trimethylcyclohex-3-en-1-yl) butan-1-one (HaloScent® D) as determined by chromatography-mass spectrometry (GC-MS) are given for several aldehydic perfumery raw materials of group C (less efficient). The release of hydrogen sulfide is expressed relative to the hydrogen sulfide release of the control perfume composition (containing 15 wt. % HaloScent® D and without any aldehydic perfumery raw material added). It can be observed from FIG. 3 that with the aldehydic perfumery raw materials of group C significantly less reduction of the formation of hydrogen sulfide could be achieved compared to the aldehydic perfumery raw materials of group A/B.

### Example 2

### Reducing the Formation of Hydrogen Sulfide in a Perfumed Shampoo Base:

A transparent shampoo base having the ingredients as given in Table 2 has been prepared.

A typical unperfumed transparent isotropic shampoo formulation is listed in Table 2. The unperfumed shampoo formulation was prepared by dispersing Polyquaternium-10 in water. The remaining ingredients of Phase A were mixed separately by addition of one after the other while mixing well after each adjunction. This pre-mix was added to the Polyquaternium-10 dispersion and mixed for another 5 min. Then, the premixed Phase B and the premixed Phase C were added (Monomuls® 90L-12 is heated to melt in Texapon® NSO IS) while agitating. Phase D and Phase E were added while agitating. The pH was adjusted with a citric acid solution to 4.0-4.5 to give the unperfumed shampoo formulation listed in Table 2.

TABLE 2
Shampoo base composition

| | Shampoo | |
|---|---|---|
| | Ingredients / INCI name | wt. % |
| A | Water deionized | 42.0 |
| | Polyquaternium-10¹⁾ | 0.3 |
| | Glycerin 85%²⁾ | 1 |
| | DMDM Hydantoin³⁾ | 0.2 |
| B | Sodium Laureth Sulfate⁴⁾ | 28 |
| | Cocamidopropyl Betaine⁵⁾ | 3.2 |
| | Disodium Cocoamphodiacetate⁶⁾ | 4 |
| | Ethoxy (20) Stearyl Alcohol | 1 |
| C | Sodium Laureth Sulfate⁴⁾ | 3 |
| | Glyceryl Laureate⁷⁾ | 0.2 |
| D | Water deionized | 1 |
| | Sodium Methylparaben⁸⁾ | 0.1 |
| E | Sodium Chloride 10% aqueous sol. | 15 |
| | Citric acid 10% aqueous sol. till pH 4.0-4.5 | q.s. |
| | Perfume | 1.0 |
| | Total | 100 |

¹⁾Ucare Polymer JR-400, Noveon
²⁾Schweizerhall
³⁾Glydant, Lonza
⁴⁾Texapon NSO IS, Cognis
⁵⁾Tego Betain F 50, Evonik
⁶⁾Amphotensid GB 2009, Zschimmer & Schwarz
⁷⁾Monomuls 90 L-12, Gruenau
⁸⁾Nipagin Monosodium, NIPA

Further, a perfume composition A according to Table 3 has been prepared.

TABLE 3
Composition of perfume A

| Perfumery raw material | Qty (g) |
|---|---|
| 1,1-dimethyl-2-phenylethyl acetate | 2.20 |
| 3,7-dimethyl-6-octenyl acetate | 16.59 |
| (+−)-1,5-dimethyl-1-vinyl-4-hexenyl | 10.72 |
| acetate | |
| 2-(6,6-dimethyl-bicyclo[3.1.1]hept- | 7.97 |
| 2-en-2-yl)ethyl acetate | |
| (+−)-2-(4-methyl-3-cyclohexen-1-yl)- | 2.11 |
| 2-propanyl acetate | |
| Tricyclo[5.2.1.0^{2,6}]dec-3/4-en-8-yl | 2.89 |
| acetate | |
| Dodecanal | 0.07 |
| (2E)-2-benzylideneoctanal | 13.94 |
| (+−)-Ethyl 2-methylpentanoate | 0.26 |
| Benzyl benzoate | 8.19 |
| Allyl (cyclohexyloxy)acetate | 2.14 |
| Methyl 2-((1RS,2RS)-3-oxo-2- | 11.94 |
| pentylcyclopentyl)acetate | |
| Hexyl 2-methylpropanoate | 2.63 |
| 2-{2-[4-methyl-3-cyclohexen-1- | 10.34 |
| yl]propyl}cyclopentanone | |
| 2-methyl-4-propyl-1,3-oxathiane) | 0.08 |
| (−)-(2S,4R)-4-methyl-2-(2-methyl-1- | 0.44 |
| propen-1-yl)tetrahydro-2H-pyran | |
| Tricyclo[5.2.1.0(2,6)]dec-3/4-en-8-yl | 4.33 |
| propanoate | |
| (3E)-4-(2,6,6-trimethyl-1- | 0.52 |
| cyclohexen-1-yl)-3-buten-2-one | |
| (3Z)-3-hexen-1-yl (3Z)-3-hexenoate | 1.24 |
| 2,4-dimethyl-3-cyclohexene-1- | 1.42 |
| carbaldehyde | |

¹ Origin: Firmenich SA, Geneva, Switzerland
²Cyclopentaneacetic acid, 3-oxo-2-pentyl-, methyl ester; origin and trademark from Firmenich SA, Geneva, Switzerland
³ Origin: Firmenich SA, Geneva, Switzerland

Then, 3-(dodecylthio)-1-(2,6,6-trimethylcyclohex-3-en-1-yl) butan-1-one (HaloScent® D) and triethanolamine have been introduced into perfume composition A at a level of 20 wt. % (HaloScent® D) and 4 wt. % (triethanolamine), respectively, to produce control perfume composition B.

Moreover, the aldehydes as given in Table 4 have been introduced into perfume A in the amounts indicated in Table 4 next to 20 wt % HaloScent® D and 4 wt. % triethanolamine to produce perfume test compositions C to G.

TABLE 4
Compositions of perfume compositions B to G

| | | Aldehydes | Perfume A | HaloScent ® | |
|---|---|---|---|---|---|
| | | dosage | dosage | D dosage | Triethanolamine |
| Perfume | Aldehydes | (wt. %) | (wt. %) | (wt. %) | dosage (wt. %) |
| B | Control without | — | 76% | 20% | 4% |
| | aldehydes | | | | |
| C | octanal | 1.5% | 74.5% | 20% | 4% |
| D | nonanal | 1.5% | 74.5% | 20% | 4% |
| E | 10-undecenal | 1.5% | 74.5% | 20% | 4% |
| F | 10-undecenal and | 2.5% | 73.5% | 20% | 4% |
| | 9-undecenal) | | | | |
| G | (+−)-2- | 2.5% | 73.5% | 20% | 4% |
| | methylundecanal | | | | |

The individual perfume compositions B to G were then dosed at a level of 1 wt. % into the shampoo base composition given in Table 2. The individual shampoo samples were then stored at room temperature and 50° C. (accelerated storage conditions), respectively, and the hydrogen sulfide malodor has been olfactively evaluated after 1 week and after 2 weeks of storage. The results are given in Table 5.

From Table 5, it can be observed that for the control shampoo sample comprising perfume composition B without aldehydes, a medium hydrogen sulfide malodor could be perceived after 1 and 2 weeks of storage at room temperature. Moreover, after 1 and 2 weeks of storage at 50° C., even a strong hydrogen sulfide malodor could be perceived.

By contrast, for the shampoo samples that comprised one of perfume compositions C to G (with individual aldehydes), no hydrogen sulfide malodor could be detected neither upon storage at room temperature nor upon storage at 50° C. This demonstrates that the individual aldehydes comprised within perfume compositions C to G effectively reduced the formation of hydrogen sulfide upon storage of the shampoo samples.

TABLE 5
Olfactory evaluation of shampoo samples upon
storage at room temperature and at 50° C.

| Perfume composition in | | | | |
|---|---|---|---|---|
| shampoo sample | 1 wk/RT | 1 wk/50° C. | 2 wk/RT | 2 wk/50° C. |
| B | Medium H₂S | Strong H₂S | Medium H₂S | Strong H₂S |
| | malodor | malodor | malodor | malodor |
| C | not det. | not det. | not det. | not det. |
| D | not det. | not det. | not det. | not det. |
| E | not det. | not det. | not det. | not det. |
| F | not det. | not det. | not det. | not det. |
| G | not det. | not det. | not det. | not det. |

Not det. = H₂S malodour not detected

### Example 3

### Reducing the Formation of Hydrogen Sulfide in a Perfumed Shampoo Base:

A mixture of 4-(dodecylthio)-4-(2,6,6-trimethylcyclohex-2-en-1-yl) butan-2-one and 4-(dodecylthio)-4-(2,6,6-trimethylcyclohex-1-en-1-yl) butan-2-one (HaloScent® I) and triethanolamine have been introduced into the perfume composition A of Table 3 at a level of 10 wt. % (HaloScent® I) and 4 wt. % (triethanolamine), respectively, to produce control perfume composition H.

Further, perfume compositions I to L have been produced by introducing HaloScent® | (10 wt. %) and triethanolamine (4 wt. %) together with various aldehydes into the perfume composition A according to Table 3. Perfume compositions I to L are shown in Table 6 below.

TABLE 6
Compositions of perfume compositions H to L

| | | | Perfume A | | |
|---|---|---|---|---|---|
| | | Aldehydes | dosage | HaloScent ® I | Triethanolamine |
| Perfume | Aldehydes | dosage (%) | (wt, %) | dosage (wt, %) | dosage (wt, %) |
| H | Control | — | 86% | 10% | 4% |
| | without | | | | |
| | aldehydes | | | | |
| I | octanal | 1.5% | 84.5% | 10% | 4% |
| J | nonanal | 1.5% | 84.5% | 10% | 4% |
| K | 10- | 2.5% | 83.5% | 10% | 4% |
| | undecenal | | | | |
| | and 9- | | | | |
| | undecenal | | | | |
| L | (+−)-2,6- | 2.5% | 83.5% | 10% | 4% |
| | dimethyl-5- | | | | |
| | heptenal | | | | |

The individual perfume compositions H to L were then dosed at a level of 1 wt. % into the shampoo base composition given in Table 2. The individual shampoo samples were then stored at room temperature and 50° C. (accelerated storage conditions), respectively, and the hydrogen sulfide malodor has been olfactively evaluated after 1 week and after 2 weeks of storage. The results are given in Table 7.

From Table 7 it can be observed that for the control shampoo sample comprising perfume composition H without aldehydes, a medium hydrogen sulfide malodor could be perceived after 1 and 2 weeks of storage at room temperature. Moreover, after 1 and 2 weeks of storage at 50° C., even a strong hydrogen sulfide malodor could be perceived.

By contrast, for the shampoo samples that comprised one of perfume compositions I to L (with individual aldehydes), no hydrogen sulfide malodor could be detected neither upon storage at room temperature nor upon storage at 50° C. This demonstrates that the individual aldehydes comprised within perfume compositions I to L effectively reduced the formation of hydrogen sulfide upon storage of the shampoo samples.

TABLE 7
Olfactory evaluation of shampoo samples upon
storage at room temperature and at 50° C.

| Perfume composition in | | | | |
|---|---|---|---|---|
| shampoo sample | 1 wk/RT | 1 wk/50° C. | 2 wk/RT | 2 wk/50° C. |
| H | Medium H₂S | Strong H₂S | Medium H₂S | Strong H₂S |
| | malodor | malodor | malodor | malodor |
| I | not det. | not det. | not det. | not det. |
| J | not det. | not det. | not det. | not det. |
| K | not det. | not det. | not det. | not det. |
| L | not det. | not det. | not det. | not det. |

Not det. = H₂S malodour not detected

### Example 4

### Reducing the Formation of Hydrogen Sulfide in a Perfume Composition:

Alpha-trimethylsiloxane-omega-trimethylsilane poly {[{{3-((2-methyl-3-oxo-5-(prop-1-en-2-yl)cyclohexyl) thio) propyl}methyl) siloxane]-co-dimethylsiloxane}) (profragrance of formula (III)) has been introduced into the perfume composition A of Table 3 at a level of 10 wt. % to produce control perfume composition M.

Further, perfume compositions N to R have been produced by introducing profragrance of formula (III) (10 wt. %) together with various aldehydes into the perfume composition A according to Table 3. Perfume compositions N to R are shown in Table 8 below.

TABLE 8
Compositions of perfume compositions M to R

| | | Aldehydes | Perfume A | profragrance of formula (III) |
|---|---|---|---|---|
| Perfume | Aldehydes | dosage (%) | dosage (wt, %) | dosage (wt,%) |
| M | Control | — | 90% | 10% |
| | without | | | |
| | aldehydes | | | |
| N | (+−)-2,6- | 1.5% | 88.5% | 10% |
| | dimethyl-5- | | | |
| | heptenal | | | |
| O | octanal | 1.5% | 88.5% | 10% |
| P | Accord 1¹ | 2.5% | 87.5% | 10% |
| Q | Accord 1¹ | 5% | 85% | 10% |
| R | Accord 2² | 5% | 85% | 10% |

¹octanal (16.67%), undecanal (16.67%), 10-undecenal (16.67%), 10-undecenal/9-undecenal (16.67%), (+/−)-2-methylundececanal (16.67%), and (+/−)-2,6-dimethyl-5-heptenal (16.67%).
²octanal (20%), undecanal (20%), 10-undecenal (20%), 10-undecenal/9-undecenal (20%), and (+/−)-2-methylundececanal (20%).

The individual perfume compositions M to R were then stored at room temperature and 50° C. (accelerated storage conditions), respectively, and the hydrogen sulfide malodor has been olfactively evaluated after 3 days and 1 week. The results are given in Table 9.

From Table 9 it can be observed that for the control perfume composition sample comprising perfume composition M without aldehydes, a strong hydrogen sulfide malodor could be perceived after three days and after 1 week at 50° C.

By contrast, for the perfume composition samples that comprised one of perfume compositions N to R (with individual aldehydes), no hydrogen sulfide malodor could be detected upon storage at 50° C. This demonstrates that the individual aldehydes comprised within perfume compositions N to R effectively reduced the formation of hydrogen sulfide upon storage of the perfume composition samples.

TABLE 9
Olfactory evaluation of perfume composition samples
upon storage at room temperature and at 50° C.

| Perfume composition in | | | | |
|---|---|---|---|---|
| shampoo sample | 3 days/RT | 3 days/50° C. | 1 wk/RT | 1wk/50° C. |
| M | not det. | Strong H₂S | not det. | Strong H₂S |
| | | malodor | | malodor |
| N | not det. | not det. | not det. | not det. |
| O | not det. | not det. | not det. | not det. |
| P | not det. | not det. | not det. | not det. |
| Q | not det. | not det. | not det. | not det. |
| R | not det. | not det. | not det. | not det. |

Not det. = H₂S malodour not detected

### Example 5

### Liquid Softener Composition:

Liquid softener compositions were prepared by introducing the perfume compositions M to R as given in Example 4 into a softener composition according to Table 10.

TABLE 10
Softener composition

| Ingredients | Amount (% wt) | Function |
|---|---|---|
| Methyl bis[ethyl(tallowate)] - | 12.2 | Softening agent |
| 2-hydroxyethyl ammonium | | |
| methyl sulfate¹⁾ | | |
| Calcium chloride sol 10% | 0.4 | Viscosity modifier |
| Water | 87.4 | |

¹⁾Stepantex ® VL 90A- Stepan
² Proxel ® GXL - Arch

The softener is prepared by weighting methyl bis[ethyl(tallowate)]-2-hydroxyethyl ammonium methyl sulfate and heating it at 65° C. Then, water is placed in the reactor and heated at 65° C. under stirring. Afterwards, the methyl bis[ethyl(tallowate)]-2-hydroxyethyl ammonium methyl sulfate is added to said mixture. The mixture is stirred for 15 minutes and CaCl₂) was added. Then, the respective perfume composition is added (0.3 to 3% by weight relative to the total weight of the liquid softener). The mixture is stirred for 15 minutes and is allowed to cool down to room temperature under stirring (viscosity: 35+/−5 mPas at a shear rate of 106 sec⁻¹).

### Example 6

### Reducing the Formation of Hydrogen Sulfide in a Shower-Gel Formula:

A transparent shower gel base having the ingredients according to Table 11 was prepared. A typical unperfumed transparent shower-gel formulation was prepared by mixing the ingredients of each phase as indicated in Table 11, one after the others, until their complete solubilization. The final pH was adjusted to 4.5 and the viscosity to 3000cPo+/−1500cPo (Brookfield RV/Spindle #4/20 RPM).

TABLE 11
Shower-gel base composition

| Phase | Ingredients | Amount (% wt.) | Function |
|---|---|---|---|
| A | WATER deionized | 52.40 | Solvent |
| | Tetrasodium EDTA¹⁾ | 0.10 | Chelating agent |
| | Sodium Benzoate | 0.50 | Preservative |
| | Propylene Glycol | 2.00 | Humectant |
| B | Sodium C12-C15 Pareth Sulfate²⁾ | 35.00 | Surfactant |
| | Cocamidopropyl Betaine³⁾ | 8.00 | Surfactant |
| | Polyquaternium-7⁴⁾ | 0.20 | Conditioning agent |
| C | Citric Acid (40%) | 1.00 | pH adjuster |
| | Sodium Chloride | 0.80 | Viscosity adjuster |

¹⁾EDETA B POWDER; trademark and origin: BASF
²⁾ZETESOL AO 328 U; trademark and origin: ZSCHIMMER & SCHWARZ
³⁾TEGO-BETAIN F 50; trademark and origin: GOLDSCHMIDT
⁴⁾MERQUAT 550; trademark and origin: LUBRIZOL

Further, a perfume composition S according to Table 12 was prepared.

TABLE 12
Composition of perfume S

| Perfumery raw material | Concentration (%) |
|---|---|
| (+)-Limonene | 41.03 |
| (+−)-2,6-dimethyl-7-octen- | 5.58 |
| 2-ol | |
| (+−)-3,7-dimethyl-1,6- | 9.34 |
| octadien-3-ol | |
| Benzyl acetate | 2.55 |
| 2-(2-methyl-2- | 1.30 |
| propanyl)cyclohexyl | |
| acetate | |
| Tricyclo[5.2.1.02,6]dec- | 1.26 |
| 3/4-en-8-yl acetate | |
| 1,1-dimethyl-2- | 2.38 |
| phenylethyl butanoate | |
| (+−)-5-heptyldihydro- | 1.23 |
| 2(3H)-furanone | |
| (+−)-2-ethyl-4-(2,2,3- | 0.93 |
| trimethyl-3-cyclopenten- | |
| 1-yl)-2-buten-1-ol | |
| Methyl 2-((1RS,2RS)-3- | 17.49 |
| oxo-2- | |
| pentylcyclopentyl)acetate | |
| (2E)-2- | 7.01 |
| benzylideneoctanal | |
| 1,4- | 9.90 |
| dioxacycloheptadecane- | |
| 5,17-dione | |

Then, 3-(dodecylthio)-1-(2,6,6-trimethylcyclohex-3-en-1-yl) butan-1-one (HaloScent® D) and triethanolamine have been introduced into perfume composition S at a level of 10 wt. % (HaloScent® D) and 4 wt. % (triethanolamine), respectively, to produce control perfume composition A′.

Then, the aldehydes as given in Table 13 have been introduced into perfume S in the amounts indicated in Table 13 next to 10 wt. % HaloScent® D and 4 wt. % triethanolamine to produce perfume test compositions A′ to F′.

TABLE 13
Compositions of perfume compositions A′ to F′

| | | Aldehydes | Perfume S | HaloScent D | |
|---|---|---|---|---|---|
| | | dosage | dosage | dosage | Triethanolamine |
| Perfume | Aldehydes | (wt. %) | (wt, %) | (wt, %) | dosage (wt, %) |
| A | Control without | — | 86.0 | 10.0 | 4.0 |
| | aldehydes | | | | |
| B | undecanal | 1.5% | 84.5 | 10.0 | 4.0 |
| C′ | octanal | 1.5% | 84.5 | 10.0 | 4.0 |
| D′ | 10- | 2.5% | 83.5 | 10.0 | 4.0 |
| | undecenal/9- | | | | |
| | undecenal | | | | |
| E | (+/−)-2- | 2.5% | 83.5 | 10.0 | 4.0 |
| | methylundecan | | | | |
| | al | | | | |
| F′ | (+/−)-2,6- | 2.5% | 83.5 | 10.0 | 4.0 |
| | dimethyl-5- | | | | |
| | heptenal | | | | |

The individual perfume compositions A′ to F′ were then dosed at a level of 1.0 wt. % into the shower-gel base composition given in Table 11. The individual perfumed shower-gel samples were then stored at 50° C. (accelerated storage conditions) and the hydrogen sulfide malodor was olfactively evaluated after 1 week of storage. The results are given in Table 14.

From Table 14 it can be observed that for the control shower-gel sample comprising perfume composition A′ without aldehydes, a strong hydrogen sulfide malodor could be perceived after 1 week of storage at 50° C.

By contrast, for the perfumed shower-gel samples that comprised one of perfume compositions B′ to F′ (with individual aldehydes), no hydrogen sulfide malodor could be detected upon storage at 50° C. for 1 week. This demonstrates that the individual aldehydes comprised within perfume compositions B′ to F′ effectively reduced the formation of hydrogen sulfide upon storage of the shower-gel samples.

TABLE 14
Olfactory evaluation of shower-gel samples upon storage at 50° C.

| Perfume composition in | |
|---|---|
| shower-gel sample | 1 wk/50° C. |
| A | Strong H₂S malodor |
| B | not det. |
| C | not det. |
| D′ | not det. |
| E | not det. |
| F | not det. |

Not det. = H₂S malodour not detected.

1. A perfume composition comprising at least one sulfur-containing pro-fragrance compound and at least one aldehydic perfumery raw material, wherein the at least one aldehydic perfumery raw material is present in an amount effective to reduce the formation of hydrogen sulfide from the sulfur-containing pro-fragrance compound, and
wherein the at least one aldehydic perfumery raw material has a chemical structure selected from the group consisting of R—CH₂—CHO and R′—CH(R′)—CHO;
wherein R represents a hydrogen atom, or a C₁ to C₁₈ hydrocarbon group, optionally comprising one to three oxygen atoms and/or one to two nitrogen atoms and/or one sulfur atom:
wherein each R′, independently from each other, represents a C₁ to C₁₈ hydrocarbon group, optionally comprising one to three oxygen atoms and/or one to two nitrogen atoms and/or one sulfur atom; or both R′, when taken together, form a C₅₋₁₆ cycloalkyl, C₅₋₁₆ cycloalkenyl, C₄₋₁₄ heterocycloalkyl or C₄₋₁₄ heterocycloalkenyl group, each optionally substituted with one or more of a C₁₋₁₅ alkyl, C₂₋₁₅ alkenyl, C₁₋₁₅ alkoxy, C₃₋₁₅ cycloalkyl, C₅₋₁₅ cycloalkenyl, C₆₋₁₀ aryl and/or C₆₋₁₀ aryloxy group, each optionally substituted with one or more of a C₁₋₈ alkyl, C₁₋₈ alkoxy, carboxylic acid and/or C₁₋₄ carboxylic ester group, wherein the heteroatom represents one or more of an oxygen atom.

2. The perfume composition of claim 1, wherein the sulfur-containing pro-fragrance compound is of formula 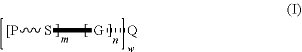
wherein:
a) w represents an integer from 1 to 10000;
b) n represents 1 or 0;
c) m represents an integer from 1 to 6:
d) P represents a hydrogen atom or a radical susceptible of generating an odoriferous
α,β-unsaturated ketone, aldehyde or carboxylic ester and is represented by the formula 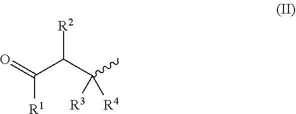
in which the wavy line indicates the location of the bond between said P and S:
R¹ represents a hydrogen atom, a C₁ to C₆ alkoxyl radical or a C₁ to C₁₅ linear, cyclic or branched alkyl, alkenyl or alkadienyl radical, optionally substituted by one to four C₁ to C₄ alkyl groups; and
R², R³ and R⁴ represent independently of each other a hydrogen atom, an aromatic ring, or a C₁ to C₁₅ linear, cyclic or branched alkyl, alkenyl or alkadienyl radical, optionally substituted by C₁ to C₄ alkyl groups: or two, or three, of the groups R¹ to R⁴ are bonded together to form a saturated or unsaturated ring having 5 to 20 carbon atoms and including the carbon atom to which said R¹, R², R³ or R⁴ groups are bonded, this ring being optionally substituted by C₁ to C₈ linear, branched or cyclic alkyl or alkenyl groups; and with the proviso that at least one of the P groups is of the formula (II);
e) G represents a multivalent radical (with a m+1 valence) derived from a cyclic, linear, alicyclic or branched alkyl, cyclic, linear, alicyclic or branched alkenyl, phenyl, alkylphenyl or alkenylphenyl hydrocarbon radical having from 1 to 22 carbon atoms, said hydrocarbon radical being possibly substituted and containing from 1 to 10 functional groups selected from the group consisting of halogens, alcohols, ethers, esters, ketones, aldehydes, carboxylic acids, thiols, thioethers, amines, quaternary amines and amides; and
f) Q represents a hydrogen atom (in which case w=1 and n=1), or represents a polymer or co-polymer selected from the group consisting of poly(alkylimine)s, peptides, lysine, polysaccharides selected from the group consisting of cellulose, cyclodextrins and starches, cationic quaternised silicon polymers, and a polymer or random co-polymer derived from monomeric units selected from the group consisting of the formulae A-1), A-2), A-3), B-1), B-2), C-1), C-2), and C-3): 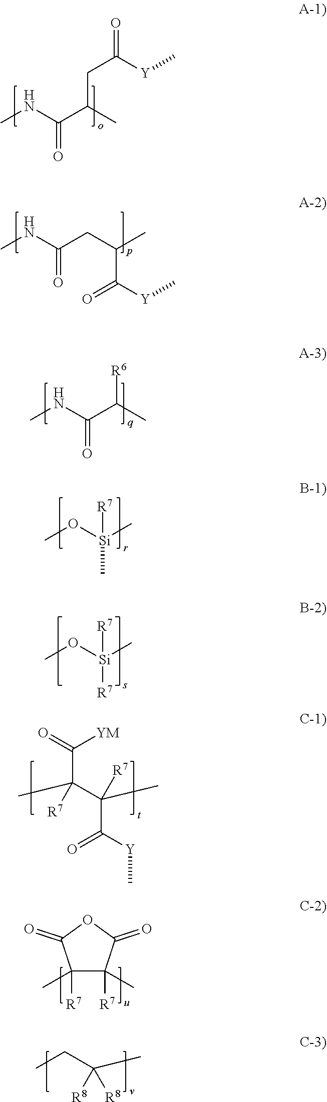
wherein the hatched lines indicate the location of the bond between said monomeric unit and G;
Y represents an oxygen or sulfur atom or a NR⁷ group;
o, p, q, r, s, t, u and v all represent independent of each other fractions between 0 and 1, with o+p+q=1, r+s=1 and t+u+v=1 and with the proviso that either o or p, as well as r and t are not equal to 0;
R⁶ represents a hydrogen atom or a side chain from a natural or unnatural amino acid, glycine, alanine, phenylalanine, arginine, histidine, lysine, aspartic acid, glutamic acid, cysteine, methionine, glutamine, asparagine, threonine, serine, leucine, isoleucine, valine, tyrosine or tryptophan;
R⁷ represents, simultaneously or independently, a hydrogen atom or a C₁-C₁₆ hydrocarbon group;
R⁸ represents, simultaneously or independently of each other
a hydrogen or halide atom;
a C₁-C₆ hydrocarbon group optionally comprising from 1 to 4 heteroatoms selected from the group consisting of oxygen and sulfur atoms;
a carboxylic group of formula COOR*, wherein R* represents a hydrogen atom, a C₁-C₆₀ alkyl or alkenyl group optionally comprising from 1 to 30 oxygen atoms;
a OR⁷ group or a COR⁷ group; or
a pyrrolidone unit, connected by the nitrogen atom; and
M represents a hydrogen atom, an alkali or earth alkali metal ion.

3. The perfume composition of claim 1, wherein the sulfur-containing pro-fragrance compound is a compound selected from the group consisting of formulae 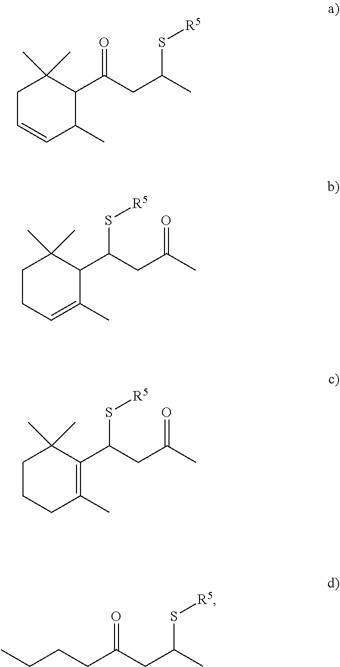
and any combination thereof;
wherein R⁵ represents a C₁-C₂₀ alkyl or alkenyl group; or
wherein the sulfur-containing pro-fragrance compound is a linear polysiloxane co-polymer comprising at least one repeating unit of formula 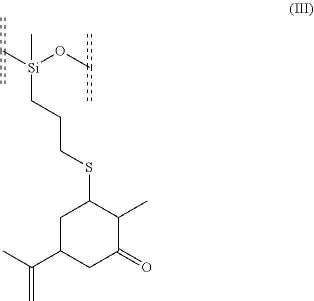
wherein the double hatched lines indicate the bonding to another repeating unit.

4. The perfume composition of claim 1, wherein the sulfur-containing pro-fragrance compound is selected from the group consisting of 3-(dodecylthio)-1-(2,6,6-trimethylcyclohex-3-en-1-yl) butan-1-one, 4-(dodecylthio)-4-(2,6,6-trimethylcyclohex-1-en-1-yl) butan-2-one, 4-(dodecylthio)-4-(2,6,6-trimethylcyclohex-2-en-1-yl) butan-2-one, and any mixture thereof.

5. The perfume composition of claim 1, wherein the perfume composition comprises the at least one sulfur-containing pro-fragrance compound in an amount from 0.05 to 50 wt. %.

6. The perfume composition of claim 1, wherein the at least one aldehydic perfumery raw material is selected from the group consisting of (+/−)-2-methyl-3-[4-(2-methyl-2-propanyl)phenyl]propanal, (+/−)-2-methylundecanal, 2,4-dimethyl-3-cyclohexene-1-carbaldehyde, (+/−)-3-(4-isopropylphenyl)-2-methylpropanal, (+/−)-3-(1,3-benzodioxol-5-yl)-2-methylpropanal, (+/−)-3/4-(4-methyl-3-penten-1-yl)-3-cyclohexene-1-carbaldehyde, (E)-2-dodecenal, (Z)-3-dodecenal, dodecanal, (+/−)-2,6-dimethyl-5-heptenal, (+/−)-3/4-(4-hydroxy-4-methylpentyl)-3-cyclohexene-1-carbaldehyde, (+/−)-3-(4-methoxyphenyl)-2-methylpropanal, (+/−)-8alpha/9alpha-methoxy-1 alpha,2beta, 6beta-tricyclo[5.2.1.02.6]decane-3beta-carbaldehyde, 3,5,6-trimethyl-3-cyclohexene-1-carbaldehyde, 2,4,6-trimethyl-3-cyclohexene-1-carbaldehyde, (+/−)-2-methyldecanal, (+/−)-2,6,10-trimethyl-9-undecenal, (1RS,6RS)-3,6-dimethyl-3-cyclohexene-1-carbaldehyde, (1RS,6RS)-4,6-dimethyl-3-cyclohexene-1-carbaldehyde, (1RS,6SR)-4,6-dimethyl-3-cyclohexene-1-carbaldehyde, (+/−)-8,8-dimethyl-1,2,3,4,5,6,7,8-octahydro-2-naphthalenecarbaldehyde, (+/−)-5,5-dimethyl-1,2,3,4,5,6,7,8-octahydro-2-naphthalenecarbaldehyde, 3-(1,1/3,3-dimethyl-2,3-dihydro-1H-inden-5-yl) propanal, decenal, dodecanal, undecanal, octanal, (+/−)-7-hydroxy-3,7-dimethyloctanal, 10-undecenal, nonanal, 10-undecenal, (9E)-9-undecenal, (9Z)-9-undecenal, 3-(1,1/3,3-dimethyl-2,3-dihydro-1H-inden-5-yl) propanal, (+/−)-3-(3-isopropyl-1-phenyl) butanal, 3-(4-tert-butylphenyl) propanal, (+)—(R)-3,7-dimethyl-6-octenal, (+/−)-3,7-dimethyl-6-octenal, (+/−)-3-phenylbutanal, 3-[(1R)-4-methyl-3-cyclohexen-1-yl)butanal, (4E)-4-methyl-5-(4-methylphenyl)-4-pentenal, 3-(4,4-dimethyl-1-cyclohexen-1-yl) propanal, phenylacetaldehyde, hexanal, (E)-4-decenal, 7-hydroxy-3,7-dimethyloctanal, (4Z)-4-dodecenal, tridecanal, heptanal, 3-phenylpropanal, 3-methylbutanal, (4-methylphenyl) acetaldehyde, 6-methoxy-2,6-dimethyloctanal, 7-isopropyl-5-methylbicyclo[2.2.2]oct-5-ene-2-carbaldehyde, 8-isopropyl-6-methylbicyclo[2.2.2]oct-5-ene-2-carbaldehyde, 3-(4-isobutylphenyl)-2-methylpropanal, octahydro-1H-4,7-methanoindene-2-carbaldehyde, (E)-2,6,10-trimethylundeca-5,9-dienal, (E)-3,7,11-trimethyldodeca-6,10-dienal, 4-(octahydro-5H-4,7-methanoinden-5-ylidene) butanal, 3-(4-isopropylphenyl) propanal, 4,8-dimethyldeca-4,9-dienal, (E)-5,9-dimethyldeca-4,8-dienal, 2-((3,7-dimethylocta-2,6-dien-1-yl)oxy) acetaldehyde, (4Z,7Z)-deca-4,7-dienal, (S,Z)-6-(2,2,3-trimethylcyclopent-3-en-1-yl) hex-4-enal, 6,8-dimethylnon-7-enal and any mixture thereof.

7. The perfume composition of claim 6, wherein the at least one aldehydic perfumery raw material is selected from the group consisting of 3-(1,1/3,3-dimethyl-2,3-dihydro-1H-inden-5-yl) propanal, decenal, dodecanal, undecanal, octanal, (+/−)-7-hydroxy-3,7-dimethyloctanal, 10-undecenal, nonanal, 10-undecenal, (9E)-9-undecenal, (9Z)-9-undecenal, 3-(1,1/3,3-dimethyl-2,3-dihydro-1H-inden-5-yl) propanal, (+/−)-3-(3-isopropyl-1-phenyl) butanal, 3-(4-tert-butylphenyl) propanal, (+)-(r)-3,7-dimethyl-6-octenal, (+/−)-3,7-dimethyl-6-octenal, (+/−)-3-phenylbutanal, 3-[(1R)-4-methyl-3-cyclohexen-1-yl)butanal, (4E)-4-methyl-5-(4-methylphenyl)-4-pentenal, 3-(4,4-dimethyl-1-cyclohexen-1-yl) propanal, phenylacetaldehyde, hexanal, (E)-4-decenal, 7-hydroxy-3,7-dimethyloctanal, (4Z)-4-dodecenal, tridecanal, heptanal, 3-phenylpropanal, 3-methylbutanal, (4-methylphenyl) acetaldehyde, (E)-3,7,11-trimethyldodeca-6,10-dienal, 4-(octahydro-5H-4,7-methanoinden-5-ylidene) butanal, 3-(4-isopropylphenyl) propanal, 4,8-dimethyldeca-4,9-dienal, (E)-5,9-dimethyldeca-4,8-dienal, 2-((3,7-dimethylocta-2,6-dien-1-yl)oxy) acetaldehyde, (4Z,7Z)-deca-4,7-dienal, (S,Z)-6-(2,2,3-trimethylcyclopent-3-en-1-yl) hex-4-enal, 6.8-dimethylnon-7-enal and any mixture thereof.

8. The perfume composition of claim 1, wherein the perfume composition comprises the at least one aldehydic perfumery raw material in an amount of at least 0.75 wt. % based on the total weight of the perfume composition.

9. The perfume composition of claim 1, wherein the perfume composition comprises a hydrogen sulfide scavenger.

10. A consumer product comprising the perfume composition according to claim 1.

11. The consumer product of claim 10, wherein the consumer product is a personal care product or home care product.

12. The consumer product of claim 10, wherein the consumer product has an acidic pH value.

13. The consumer product of claim 10, wherein the consumer product comprises a preservative.

14. A method for reducing the formation of hydrogen sulfide from a sulfur-containing pro-fragrance compound, the method comprising adding at least one aldehydic perfumery raw material to the sulfur-containing pro-fragrance compound,
wherein the at least one aldehydic perfumery raw material has a chemical structure selected from the group consisting of R—CH₂—CHO and R′—CH(R′)—CHO;
wherein R represents a hydrogen atom, or a C₁ to C₁₈ hydrocarbon group, optionally comprising one to three oxygen atoms and/or one to two nitrogen atoms and/or one sulfur atom; and
wherein each R′, independently from each other, represents a C₁ to C₁₈ hydrocarbon group, optionally comprising one to three oxygen atoms and/or one to two nitrogen atoms and/or one sulfur atom; or both R′, when taken together, form a C₅₋₁₆ cycloalkyl, C₅₋₁₆ cycloalkenyl, C₄₋₁₄ heterocycloalkyl or C₄₋₁₄ heterocycloalkenyl group, each optionally substituted with one or more of a C₁₋₁₅ alkyl, C₂₋₁₅ alkenyl, C₁₋₁₅ alkoxy, C₃₋₁₅ cycloalkyl, C₅₋₁₅ cycloalkenyl, C₆₋₁₀ aryl and/or C₆₋₁₀ aryloxy group, each optionally substituted with one or more of a C₁₋₈ alkyl, C₁₋₈ alkoxy, carboxylic acid and/or C₁₋₄ carboxylic ester group, wherein the heteroatom represents one or more of an oxygen atom.

15. (canceled)

16. The perfume composition of claim 3, wherein R⁵ represents a C₆-C₁₅ alkyl or alkenyl group.

17. The perfume composition of claim 1, wherein the perfume composition comprises the at least one sulfur-containing pro-fragrance compound in an amount from 0.1 to 30 wt. %.

18. The perfume composition of claim 1, wherein the perfume composition comprises the at least one aldehydic perfumery raw material in an amount of at least 1.5 wt. % based on the total weight of the perfume composition.

19. The perfume composition of claim 1, wherein the perfume composition comprises a hydrogen sulfide scavenger selected from the group consisting of monoethanolamine, diethanolamine, triethanolamine, methyldiethanolamine, dimethylethanolamine, alkyldiethanolamines, ethoxylated alkyldiethanolamines, 1,1′,1″,1″-[1,2-ethanediyldi(nitrilo)]tetra(2-propanol), and any mixture thereof.

20. The consumer product of claim 10, wherein the consumer product is a fabric conditioner, a shower gel, or a rinse-off conditioner.

21. The consumer product of claim 10, wherein the consumer product has a pH below 5.5.
